SEQUENCE LISTING (10 sequences)

SEQ ID NO: 1 - protein, 504 aa, Homo sapiens
MEPAGPAPGRLGPLLCLLLAASCAWSGVAGEEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIYIVVGVVCTLLVALLMAALYLVRIRQKKAQGSTSSTRLHEPEKNAREITQDTNDITYADLNLPKGKKPAPQAAEPNNHTEYASIQTSPQPASEDTLTYADLDMVHLNRTPKQPAPKPEPSFSEYASVQVPRK

SEQ ID NO: 2 - protein, 504 aa, Homo sapiens [sequence omitted]

SEQ ID NO: 3 - protein, 508 aa, Homo sapiens
MEPAGPAPGRLGPLLCLLLAASCAWSGVAGEEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIYIVVGVVCTLLVALLMAALYLVRIRQKKAQGSTSSTRLHEPEKNAREITQVQSLDTNDITYADLNLPKGKKPAPQAAEPNNHTEYASIQTSPQPASEDTLTYADLDMVHLNRTPKQPAPKPEPSFSEYASVQVPRK

SEQ ID NO: 4 - protein, 504 aa, Homo sapiens [sequence omitted]

SEQ ID NO: 5 - protein, 508 aa, Homo sapiens; identical to SEQ ID NO: 3

SEQ ID NO: 6 - protein, 508 aa, Homo sapiens [sequence omitted]

SEQ ID NO: 7 - protein, 507 aa, Homo sapiens [sequence omitted]

SEQ ID NO: 8 - protein, 503 aa, Homo sapiens
MEPAGPAPGRLGPLLCLLLAASCAWSGVAGEEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIYIVVGVVCTLLVALLMAALYLVRIRQKKAQGSTSSTRLHEPEKNAREITQDTNDITYADLNLPKGKKPAPQAAEPNNHTEYASIQTSPQPASEDTLTYADLDMVHLNRTPKQPAPKPEPSFSEYASVQVPRK

SEQ ID NO: 9 - protein, 507 aa, Homo sapiens
MEPAGPAPGRLGPLLCLLLAASCAWSGVAGEEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIYIVVGVVCTLLVALLMAALYLVRIRQKKAQGSTSSTRLHEPEKNAREITQVQSLDTNDITYADLNLPKGKKPAPQAAEPNNHTEYASIQTSPQPASEDTLTYADLDMVHLNRTPKQPAPKPEPSFSEYASVQVPRK

SEQ ID NO: 10 - protein, 508 aa, Homo sapiens
MTLKTRCCIWTLSPALAYFILPEIHRGVAGEEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIYIVVGVVCTLLVALLMAALYLVRIRQKKAQGSTSSTRLHEPEKNAREITQVQSLDTNDITYADLNLPKGKKPAPQAAEPNNHTEYASIQTSPQPASEDTLTYADLDMVHLNRTPKQPAPKPEPSFSEYASVQVPRK

(19) United States
(12) Patent Application Publication
(10) Pub. No.: US 2022/0120731 A1
(43) Pub. Date: Apr. 21, 2022

(54) SIRPa EXPRESSION ON T CELLS IS A BIOMARKER FOR FUNCTIONAL T CELLS DURING EXHAUSTION

(71) Applicants: The Board of Trustees of the Leland Stanford Junior University, Stanford, CA (US); The United States of America, as represented by the Secretary, Department of Health and Human Servic, Bethesda, MD (US)

(72) Inventors: Michal Caspi Tal, Cupertino, CA (US); Lara Myers, Hamilton, MT (US); Kim J. HasenKrug, Victor, MT (US); Ying Ying Yiu, Concord, CA (US); Irving L. Weissman, Stanford, CA (US)

(21) Appl. No.: 17/425,224

(22) PCT Filed: Jan. 30, 2020
(86) PCT No.: PCT/US2020/015905

§ 371 Date: Jul. 22, 2021

Related U.S. Application Data
(60) Provisional application No. 62/800,333, filed on Feb. 1, 2019.

(51) Int. Cl. G01N 33/50; C12N 5/0783; A61K 35/17

(52) U.S. Cl. CPC G01N 33/505; G01N 2333/70596; A61K 35/17; C12N 5/0638

(57) ABSTRACT

Prolonged exposure of CD8⁺ T cells to antigenic stimulation leads to a state of diminished function, termed exhaustion; during exhaustion there is a subset of functional CD8⁺ T cells defined by surface expression of SIRP(alpha) protein. On SIRP⁺ CD8⁺ T cells, expression of coinhibitmy receptors is counterbalanced by expression of co-stimulatory receptors and it is only these SIRP⁺ cells that actively proliferate, transcribe IFNg and show cytolytic activity. Therapeutic blockade of PD-L1 or other inhibitory receptors to reinvigorate exhausted CD8⁺ T cells expands the cytotoxic subset of SIRP⁺ CD8⁺ T cells.

### CROSS REFERENCE

This application is a 371 application and claims the benefit of PCT Application No. PCT/US2020/015905, filed Jan. 30, 2020 which claims benefit of U.S. Provisional Patent Application No. 62/800,333 filed Feb. 1, 2019 which applications are incorporated herein by reference in their entirety.

### BACKGROUND

Key effectors in host immune responses to intracellular pathogens are CD8⁺ cytolytic T lymphocytes (CTL). CTLs become activated in a pathogen-specific manner, undergo extensive expansion, and function to locate and kill infected cells. While the destructive capacity of CTLs is essential for their activity, it also provides the potential to cause immunopathological damage. Thus, the immune system has evolved multilayered mechanisms to control the duration and magnitude of CTL responses. For example, the contraction of the CD8⁺ T cell response is hardwired and not dependent on pathogen clearance. Even in circumstances where a virus is not cleared, the CTL population nevertheless contracts. Furthermore, prolonged antigenic stimulation during chronic infections causes a diminished state of T cell function known as exhaustion. Such dysfunction protects the host from immunopathology, but also contributes to the failure to clear infections and cancer.

T cell exhaustion was first discovered in mice chronically infected with lymphocytic choriomeningitis virus (LCMV), but it is now known to also occur in humans chronically infected with viruses such as HIV and HCV. Exhausted CD8⁺ T cells have increased expression of co-inhibitory receptors whose breadth and level of expression have been correlated with dysfunction. Thus, high expression of multiple co-inhibitory receptors is considered a cardinal feature of exhausted CD8⁺ T cells. Blockade of one of these, Programmed Cell Death Protein 1 (PD-1) increases the function of exhausted CD8⁺ T cells. Cells with intermediate rather than high expression levels of PD-1 have been reported to comprise a subset of less exhausted cells whose function can be rescued by PD-1 blockade. Furthermore, simultaneous blockade of more than one co-inhibitory receptor (eg. PD-1 and LAG-3 or PD-1 and TIM-3) has a much more potent effect on enhancing CD8⁺ T cell function than blockade of a single receptor. Thus, the state of CD8⁺ T cell exhaustion is reversible and evidence indicates that not all CD8⁺ T cells become uniformly exhausted. Despite their reduced function, exhausted T cells are not inert and help maintain control over virus replication during chronic infection.

T cell exhaustion is also a factor in cancer immunotherapy. T cells can recognize tumor antigens (TAs) expressed by cancer cells and induce tumor rejection in vivo, however high-frequency TA-specific CD8⁺ T cells often fail to promote tumor regression in patients with advanced cancer. As with virus infection, T cells can fail to act due to exhaustion. The recent successes of immune checkpoint blockade with anti-CTLA-4 and anti-PD-1 mAbs in multiple cancers illustrate the potency of therapeutic strategies aiming at counteracting these immunoregulatory pathways.

Methods of identifying and treating T cell exhaustion is of great interest.

### SUMMARY

Prolonged exposure of CD8⁺ T cells to antigenic stimulation leads to a state of diminished function, termed exhaustion. It is shown herein that during exhaustion there is a subset of functional CD8⁺ T cells defined by surface expression of SIRPα protein. On SIRP+CD8⁺ T cells, expression of coinhibitory receptors is counterbalanced by expression of co-stimulatory receptors and it is only these SIRP+ cells that actively proliferate, transcribe IFNγ and show cytolytic activity. SIRP+CD8⁺ T cells are present in patients with chronic infections. Therapeutic blockade of PD-L1 or other inhibitory receptors to reinvigorate CD8⁺ T cells during chronic infection expands the cytotoxic subset of SIRP+CD8⁺ T cells.

In some embodiments, methods are provided for identifying functional CD8⁺ T cells by cell surface expression of SIRPα. In some embodiments, the functional CD8+ cells are identifying in a population of exhausted T cells. In some embodiments the T cells are positive for one or more inhibitory receptors, which include, without limitation, PD-1, CTLA-4, LAG-3, TIM-3, etc. In some embodiments the cells co-express PD-1 and SIRPα. In some embodiments the exhausted T cells are specific for a tumor antigen. In some embodiments the exhausted T cells are specific for an antigen of a pathogen in a chronic infection, which include, without limitation, viral and bacterial antigens.

In some embodiments, a patient sample is analyzed to identify if functional CD8⁺, SIRPα⁺ functional T cells are present. In some such embodiments, the patient is treated with a regimen to expand such CD8⁺, SIRPα⁺ functional T cells, which regimen may include, without limitation, blockade of inhibitory receptors. In some embodiments the patient is treated with a regimen comprising blockade of PD-1/PD-L1. The regimen may comprise administering an effective dose of an antibody that blocks PD-1/PD-L1.

A method may comprise obtaining a patient sample; analyzing the sample for the presence of CD8⁺, SIRPα⁺ functional T cells; if such functional T cells are present treating the patient with a regimen to block inhibitory receptors, which may include PD-1/PD-L1. A method may further comprise obtaining a patient sample following the regimen, to determine if there is an expansion of CD8⁺, SIRPα⁺ functional T cells.

In some embodiments, functional CD8⁺, SIRPα⁺ functional T cells are isolated from an individual, for example by affinity methods such as flow cytometry, magnetic affinity isolation, and the like. The T cells thus isolated may be provided as a substantially pure population. The T cells thus isolated can be analyzed for antigenic specificity to identify appropriate antigens for stimulation. The T cells thus isolated can be stimulated and expanded in culture, for example by contacting with suitable cytokines, antigens, and immune checkpoint blockade. The expanded T cell population can be reintroduced into the individual for therapeutic purposes, e.g. activity against cells bearing the cognate pathogen or tumor antigen. Alternatively, the cognate antigen can be provided in combination with a regimen as described above in order to stimulate the CD8⁺, SIRPα⁺ functional T cells.

In some embodiments, the patient analyzed for presence of CD8⁺, SIRPα⁺ functional T cells is infected with a chronic pathogen infection, for example including but not limited to viral infections, e.g. retrovirus, lentivirus, hepadna virus, herpes viruses, pox viruses, human papilloma viruses, etc.; intracellular bacterial infections, e.g. Mycobacterium, Chlamydophila, Ehrlichia, Rickettsia, Brucella, Legionella, Francisella, Listeria, Coxiella, Neisseria, Salmonella, Yersinia sp, etc.; and intracellular protozoan pathogens, e.g. Plasmodium sp, Trypanosoma sp., Giardia sp., Toxoplasma sp., Leishmania sp., etc.

In some embodiments, the patient analyzed for the presence of CD8⁺, SIRPα₊ functional T cells has a cancer. Examples of cancer cells include but are not limited to AML, ALL, CML, adrenal cortical cancer, anal cancer, aplastic anemia, bile duct cancer, bladder cancer, bone cancer, bone metastasis, brain cancers, central nervous system (CNS) cancers, peripheral nervous system (PNS) cancers, breast cancer, cervical cancer, childhood Non-Hodgkin's lymphoma, colon and rectum cancer, endometrial cancer, esophagus cancer, Ewing's family of tumors (e.g. Ewing's sarcoma), eye cancer, gallbladder cancer, gastrointestinal carcinoid tumors, gastrointestinal stromal tumors, gestational trophoblastic disease, Hodgkin's lymphoma, Kaposi's sarcoma, kidney cancer, laryngeal and hypopharyngeal cancer, liver cancer, lung cancer, lung carcinoid tumors, Non-Hodgkin's lymphoma, male breast cancer, malignant mesothelioma, multiple myeloma, myelodysplastic syndrome, myeloproliferative disorders, nasal cavity and paranasal cancer, nasopharyngeal cancer, neuroblastoma, oral cavity and oropharyngeal cancer, osteosarcoma, ovarian cancer, pancreatic cancer, penile cancer, pituitary tumor, prostate cancer, retinoblastoma, rhabdomyosarcoma, salivary gland cancer, sarcomas, melanoma skin cancer, non-melanoma skin cancers, stomach cancer, testicular cancer, thymus cancer, thyroid cancer, uterine cancer (e.g. uterine sarcoma), transitional cell carcinoma, vaginal cancer, vulvar cancer, mesothelioma, squamous cell or epidermoid carcinoma, bronchial adenoma, choriocarinoma, head and neck cancers, teratocarcinoma, or Waldenstrom's macroglobulinemia.

### BRIEF DESCRIPTION OF THE DRAWINGS

The invention is best understood from the following detailed description when read in conjunction with the accompanying drawings. The patent or application file contains at least one drawing executed in color. Copies of this patent or patent application publication with color drawing(s) will be provided by the Office upon request and payment of the necessary fee. It is emphasized that, according to common practice, the various features of the drawings are not to-scale. On the contrary, the dimensions of the various features are arbitrarily expanded or reduced for clarity. Included in the drawings are the following figures.

FIG. 1A-1O. PD-1 and SIRPα expression on CD8⁺ T cells during acute and chronic infection. Wildtype C57/BL6 mice were adoptively transferred with 1000 TCR transgenic lymphocytic choriomeningitis virus (LCMV)-specific CD8⁺ T cells from spleens of naïve P14 mice and then infected with either LCMV Arm or Cl13. The cells were then analyzed at multiple timepoints by microarray and the data were made publicly available. SIRPα (FIG. 1A) and PD-1 (FIG. 1B) expression were analyzed by Dunnett's multiple comparisons test with each time-point compared to time zero (n=4 mice per time point except for d6 LCMVArm, n=3. SEMs are shown as bars). Representative flow cytometry contour plots of Thy1.1-gated, adoptively transferred P14 CD8⁺ T cells at 42 days post-infection with Arm (FIG. 1C) or CL13 (FIG. 1D) are shown. Numbers in the upper right quadrant are mean percentages of SIRPα⁺ cells (n=4 Arm, n=3 Cl13), P=0.0029 by unpaired, two-way t test. (FIG. 1E) Average MFI of SIRPα expression (P=0.0088 by unpaired two-way t test). CD8+ splenocytes from naïve (FIG. 1F), 7 dpi (FIG. 1G), 14 dpi (FIG. 1H), or chronic (FIG. 1I) Friend virus infected mice were analyzed by flow cytometry for CD11a expression and FV-D^{b} gagL dextramer staining. A representative FACS plot is shown. Dextramer+CD11a⁺ (FIGS. 1J, 1K) and dextramer CD11a⁻ subsets (FIGS. 1L-1O) were further analyzed for PD-1 and SIRPα expression during the course of FV infection. Arrow originate in the quadrant further analyzed and point to the analysis. The percentage in each quadrant depicts the means from 8 mice at each time-point, with standard deviations in parentheses.

FIGS. 2A-2H. PD-1 and SIRPα expression kinetics during FV infection. Day 7 acute or chronically infected mice were adoptively transferred with 1×10⁶ bead-purified and CellTrace™ (violet)-labeled CD8⁺ T cells from the spleens of naïve Thy1.1+CD8.TCR Tg mice. The spleens of recipient mice were analyzed by flow cytometry at 72 hours post-transfer. (FIGS. 2A, 2E) Analysis of dual expression of PD-1 and SIRPα by Pearson Correlation showed highly significant correlation (P<0.0001 for both actue and chronic). Representative plots showing expression of PD-1 (FIGS. 2B, 2F) and SIRPα (FIGS. 2C, 2G) during proliferation (analyzed by dilution of CellTrace™ fluorescence) is shown on donor cells from acute and chronic recipients. Quantification of results from individual mice during acute (FIG. 2D) and chronic infection (FIG. 2H) are shown with each dot representing an individual mouse and the bar representing the mean. The 1-8 designation depicts the gating strategy for identifying individual cell divisions, but the numbers are only relative as the zero division expression of CellTrace was not evident. Data are from one of two independent experiments with similar results. Linear regression analyses were used to draw the lines in d (R²=0.6148, P=0.0369) and h (R²=0.8837, P=0.0053). The difference between the slopes of the lines for acute (m=5.781) vs chronic (m=1.836) was very significant, P=0.0088. During transfer into chronically infected mice (FIG. 2H), there was a slight but significant increase in the proportion of cells expressing SIRPα between cell division 2 (mean=23.5) and cell division 7 (mean=32.83) P=0.0241 by two-way Student's t test.

FIGS. 3A-3L. Phenotype of Friend virus-specific PD-1⁺ SIRPα⁻ and PD-1+ SIRPα⁺ CD8⁺ T cells in mice chronically infected with FV. Splenocytes from mice chronically infected with FV were analyzed by multiparameter flow cytometry for surface expression of (FIG. 3A) CD122, (FIG. 3B) Tim3, (FIG. 3C) Lag3, (FIG. 3D) CD95 (Fas), (FIG. 3E) CD43, (FIG. 3F) CD44, (FIG. 3G) CD40, (FIG. 3H) CD278 (ICOS), (FIG. 3I) KLRG1, (FIG. 3J) CD62L, (FIG. 3K) CD47, and (FIG. 3L) CX₃CR1. A representative off-set histogram overlay is displayed for each marker as well as the average geometric mean fluorescence intensity (MFI) from one experiment is given (n=4 mice). CD8₊ dextramer cells (non-DbgagL-specific cells from infected mice) are shown in dashed gray, PD-1⁺/SIRPα⁻ CD8₊ dextramer⁺ cells are shown in solid line gray and PD-1⁺/SIRPα⁺ CD8₊ dextramer⁺ are shown in black. The vertical dashed line delineates positivity relative to the FMO control). Results are from one of three independent experiments with similar results (with n=8 additional mice). ns,p>0.05, * p≤0.05, ** p≤0.01, ***p≤0.001, ****p≤0.0001 (unpaired, two way t tests).

FIG. 4. Differential gene expression of bulk sorted SIRPα⁻ or SIRPα+ transgenic CD8+PD-1⁺ T cells. CD8⁺ T cells from naïve FV-specific Thy1.1+CD8.TCR mice were transferred i.v. into Y10 mice chronically infected with FV. After 13-15 days, CD8+ cells were purified from the spleens of these recipients using anti-CD8 paramagnetic beads and the Miltenyi MACS systems. Cells were then stained with anti-Thy1.1; anti-CD8; anti-PD-1; anti-SIRPα and sorted into CD8⁺ Thy1.1+PD-1+SIRPα⁻ and CD8⁺ Thy1.1+PD-1⁺ SIRPα⁺ populations for analysis using a BD FACSAriallu. A heat map representation of the top 325 differentially expressed genes between the SIRPα⁻ and SIRPα⁺ transgenic CD8+PD-1⁺ T cells was generated using the Benjamini-Hochberg procedure to decrease the false discovery rate using DESeq2 default settings (FDR 0.1). Selected differentially expressed genes are highlighted on the left. Heat map is color coded by row z-score as shown.

FIG. 5A-5M. SIRPα expression on PD-1⁺ CD8⁺ T cells identifies cells with enhanced proliferation and cytolytic ability. CD8⁺ FV-D^{b} gagL dextramer⁺ splenocytes from 14 dpi (FIG. 5A) or chronically infected mice (FIG. 5B) were analyzed by flow cytometry for PD-1 and SIRPα expression and the gated SIRPα positive and negative subsets were analyzed for surface CD107a and intracellular granzyme B. Representative FACS plots with gating strategy are shown in (FIG. 5C-5F). Data are from two independent experiments (n=8 total mice) where each dot represents the percentage of (FIG. 5G) intracellular granzyme B, (FIG. 5H) surface CD107a and (FIG. 5I) intracellular Ki-67 from each cell subset. Mice (FIG. 5J) chronically or (FIG. 5K) acutely infected with FV were adoptively transferred with 1×10⁶ naïve FV-specific TCR transgenic CD8 T cells and then at 13-15 days post-transfer the cells were recovered and separated into PD-1⁺ SIRPα⁻ and PD-1⁺ SIRPα⁺ subpopulations by FACS cell sorting. A 2-hour in vitro cytotoxicity assay was then performed with the sorted effector cells, as described in methods. Each dot represents the background-corrected value of substrate fluorescence for an individual sample at target to effector ratios, 1:4 and 1:10. The bar is the mean. The negative control is bead purified CD8⁺ T cells from a naïve Y10 mouse and is represented by the horizontal dashed line. Data from chronic infection are from 3 independent experiments. Subsets of splenic CD8⁺ T cells from mice chronically infected with FV were analyzed by intracellular flow cytometry for TCF-1. (FIG. 5L) A representative histogram overlay is displayed, depicting the MFI of each labeled subset. (FIG. 5M) Each dot represents an MFI value of intracellular TCF-1 for a given subset. Data are from 1 of 2 independent experiments, a total of n=11 mice were analyzed. All bars in the figure represent the mean. ns,p>0.05, * p≤0.05, ** p≤0.01, ***p≤0.001, ****p≤0.0001 (unpaired, two-way t tests).

FIG. 6A-6F. Enhanced in vivo cytolytic activity against target cells expressing CD47, the ligand for SIRPα. Naive and mice chronically infected with FV were adoptively transferred with differentially fluorophore-labeled target cells as outlined in panel A and in the methods. Briefly, wildtype C57/BL6 splenocytes were differentially labeled with two concentrations of CFSE and CD47^{-/-} splenocytes were differentially labeled with two concentrations of CellTrace™-violet. The brighter of each subset was peptide-loaded with 25 μM FV-D^{b}GagL peptide, while the lower intensity subset was sham-loaded in DMSO media. (FIG. 6A) The gating strategy identifying the target cell populations and representative dot plots showing target cell populations (FIG. 6B) before injection and retrieved from (FIG. 6C) naïve and (FIG. 6D) chronic recipients 6 hours post-transfer. The percentages given are the means for each cell subset combining data from two independent experiments. (FIG. 6E, 6F) The percent killing comparing C57/BL6 WT and CD47^{-/-} target cells as described in methods. The data points showing the killing of each type of target cell within the same recipient mouse were connected with a line and the differences were statistically significant as indicated by two-way paired t tests. Data from the two independent experiments include a total of 20 mice. Virus-specific killing is defined as the percentage of killing of each population of FV peptide-pulsed cells calculated as follows: 100−([% peptide pulsed in infected/% un-pulsed in infected)/(% peptide pulsed in uninfected/% unpulsed in uninfected)]×100).

FIG. 7. Upregulation of CD47 on FV-infected cells. CD47 is upregulated on FV-infected splenocytes. Mice were infected with Friend virus or left naïve and the splenocytes were analyzed by flow cytometry to compare the expression MFI of CD47 and on naïve or FV-infected (Virus⁺) and uninfected (Virus⁻) total splenocytes at 7 days (D7) post-infection with significance as indicated by one-way ANOVA. Virus was detected by cell surface expression of glycosylated gag antigen with mAb 34 as described in the methods. Data from one of 4 independent experiments are shown.

FIG. 8A-8F. Increased SIRPα expression on CD8⁺ T cells from hepatitis C virus (HCV) infected patients identifies a more activated phenotype. (FIG. 8A) Representative plots of CYTOF analysis of the median healthy donor and (FIG. 8B) the median HCV patient donor in regards to SIRPα expression levels. (FIG. 8C) CD8⁺ CD57⁻ CD28⁺ (circles) and CD8⁺ CD57⁺ CD28⁻ (squares) PBMCs from healthy controls (open symbols) and HCV infected patients (closed symbols) were analyzed by CYTOF for SIRPα expression. For CyTOF analyses fluorescence intensity data are commonly transformed to arcsinh for analysis and display. The SIRPα⁺ and SIRPα⁻ subsets of CD8⁺ CD57⁻ CD28⁺ (circles) and CD8⁺ CD57⁺ CD28⁻ (squares) from HCV infected patients were further analyzed by CYTOF for expression of (FIG. 8D) phosphorylated STAT3, (FIG. 8E) CD244 and (FIG. 8F) HLADR. Median expression levels (Arcsinh transformed) for each subset are represented by corresponding symbols, where each symbol represents an individual sample and the bar represents the mean. Differences between samples were statistically significant as shown by two-way unpaired t test.

FIG. 9A-9B. Expansion of cytolytic FV-specific SIRPα⁺ CD8⁺ T cells during PD-L1 blockade. As described in the methods, mice chronically infected with FV were injected every other day with anti-PD-L1 blocking antibody and analyzed the second day following the final injection for the number of dextramer^{+ CD}107a⁺ (FIG. 9A) and dextramer⁺ SIRPα⁺ CD107a⁺ (FIG. 9B) T cells in the spleen. Bars represent the mean and symbols represents individual mice (n=6-7) pooled from 2 independent experiments, with statistical differences analyzed by two-way unpaired t tests.

FIG. 10. Correlation analysis. Sirpα expression significantly correlated with Pdcd1 expression at a Pearson correlation coefficient of 0.516 and p-value of 0.001263. Data from both acute and chronic LCMV infection were used. When all of the genes are organized by order of correlation with the expression pattern of Pdcd1, Sirpα ranked 586 out of 20776 genes (97^{th} percentile). The correlation significance was confirmed for multiple comparisons using the Benjamini-Hochberg procedure (<0.05, FDR<0.05).

FIG. 11A-11B. SIRPα expression on CD8⁺ T cells compared to macrophages. Splenocytes from naive and 14 dpi mice were analyzed by multiparameter flow cytometry for expression of SIRPα. (FIG. 11A) A representative histogram overlay is displayed. CD8⁺ T cells from naïve mice are shown in unfilled solid line, CD8+ dextramer+ T cells from 14 days post-FV infected mice are shown in filled gray and CD11 b+ cells (macrophages) from naive mice are shown in filled black. (FIG. 11B) MFI of SIRPα expression where the bar represents the mean, with standard deviation. Data are from one of 3 independent experiments for a total of n=7 naive and n=10 14 dpi mice. (One-Way ANOVA with Tukey's multiple comparison test).

FIG. 12A-12D. PD-1 and SIRPα expression on CD8+ T cells during FV infection. CD8+ splenocytes from (FIG. 12A) naive. (FIG. 12B) 7 dpi, (FIG. 12C) 14 dpi, or (FIG. 12D) chronic Friend virus infected mice were analyzed by flow cytometry for CD11a and FV-db gagL dextramer expression. The CD8₊ dextramer− CD11a+ were further analyzed by flow cytometry for PD-1 and SIRPα expression during the course of FV infection as shown. The percentage in each quadrant depicts the mean, with standard deviations in parentheses. Numerical data are combined from two independent experiments (n=8 mice at each time-point).

FIG. 13. Venn diagram comparing gene regulation in FV and LCMV infections. Venn was generated by finding the intersection of three gene lists: 1. genes significantly (p<0.05) correlated with Sirpα expression in T cells during various stages of LCMV infection (FIG. 1a) (in red); 2. Genes significantly (p-adj<0.1) enriched in Sirpα− T cells in FV infection (FIG. 4) (in gray); and 3. Genes significantly (p-adj<0.1) enriched in Sirpa+ T cells in FV infection (in blue).

FIG. 14A-14E. HCV-induced SIRPα expression and in vitro activation. (FIG. 14A, 14B) PBMCs from HCV patients were analyzed by flow cytometry for SIRPα expression. CD3+ CD8+ CD57− γδTCR− cells and CD3+ CD8+ CD57+ γδTCR− are shown stained for SIRPα in comparison to the full staining panel excluding anti-SIRPα (FMO) control. (FIG. 14C) Additionally, the CD57+ and CD57− subsets were compared in the histogram to the SIRPα expression levels on CD14+ cells from within the same HCV patient's PBMCs. Flow plots are a representative example. (FIG. 14D, 14E) PBMCs were labeled with CFSE and incubated in plates coated with anti-CD8 and anti-CD28 antibodies or control wells. After 5 days in vitro, SIRPα expression and CFSE dilution was analyzed by flow cytometry.

FIG. 15A-15P. Representative gating strategies for FlowJo data analysis. (FIG. 15A-15D) The initial gating strategy for FIG. 1f-i to identify endogenous FV-specific CD8+ T cells expressing PD-1 and SIRPα. (FIG. 15E-15G) The initial gating strategy for FIG. 2 to identify the adoptively transferred FV-specific CD8+ Thy1.1+T cells. (FIG. 15H-15M) The initial gating strategy for FIGS. 3 and 5 to characterize the phenotype of FV-specific CD8+ T cells expressing PD-1 and SIRPα. (FIG. 15N-15P) The initial gating strategy for FIG. 6 to identify the four target cell populations, which were all APC-labelled and then differentially labelled with either two intensities of CellTrace™ Violet or CFSE, as depicted in FIG. 6a.

### DETAILED DESCRIPTION OF EMBODIMENTS

The present invention relates to methods for identifying functional CD8⁺ T cells by cell surface expression of SIRPα. In some embodiments, the functional CD8⁺ cells are identifying in a population of exhausted T cells. In some embodiments the T cells are positive for one or more inhibitory receptors, which include, without limitation, PD-1, CTLA-4, LAG-3, TIM-3, etc. In some embodiments the cells co-express PD-1 and SIRPα. In some embodiments the exhausted T cells are specific for a tumor antigen. In some embodiments the exhausted T cells are specific for an antigen of a pathogen in a chronic infection, which include, without limitation, viral and bacterial antigens. Also provided are kits and companion diagnostics for performing the methods.

Before the present methods and kits are described, it is to be understood that this invention is not limited to particular method or composition described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

It must be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g. polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

T cell exhaustion. Exhausted T cells were originally identified in a chronic lymphocytic choriomeningitis virus (LCMV) infection model. The LCMV-specific CD8⁺ T cells expressing activation markers (CD69^{hi}D44^{hi}CD62^{low} were unable to perform the anti-viral functions. T-cell exhaustion is a state of T-cell dysfunction in chronic environment; exhausted T cells express high levels of inhibitory receptors, including programmed cell death protein 1 (PD-1), lymphocyte activation gene 3 protein (LAG-3), T-cell immunoglobulin domain and mucin domain protein 3 (TIM-3), cytotoxic T lymphocyte antigen-4 (CTLA-4), band T lymphocyte attenuator (BTLA) and T-cell immunoglobulin and immunoreceptor tyrosine-based inhibitory motif domain (TIGIT). The other principal characteristic of exhausted T cells is the loss of function in a hierarchical manner. Such functions as interleukin-2 (IL-2) production and ex vivo killing capacity are lost at the early stage of exhaustion, whereas tumor necrosis factor-α (TNF-α) production is lost at the intermediate stage, interferon-γ and granzymeB(GzmB) production are lost at the advanced stage of exhaustion.

Complete activation of T cells requires three signals, the first signal is the interaction of antigenic peptide—MHC complex with TCR, the second signal is costimulatory or co-inhibitory signal provided by antigen-presenting cells, the third signal is the stimulation by extracellular cytokines such as IL-2. Among these signals, the second signal determines the promotion or inhibition of T-cell cytokine production and effector function, appropriate co-inhibitory signals dampen inflammation to avoid tissue damage from excessive immune reaction, whereas durative and overmuch co-inhibitory signals lead to T-cell hyporesponsiveness. Co-inhibitory signals are primarily mediated by inhibitory receptors that are the major phenotypes of exhausted T cells. Consistent with chronic infection, T cells in tumor environments can also exhibit exhausted phenotype and function. Exhausted T cells in cancer express high levels of inhibitory receptors, including PD-1, CTLA-4, TIM-3, LAG-3, BTLA and TIGIT, as well as show impaired effector cytokine production, such as IL-2, TNF-α, IFN-γ and GzmB.

CD8⁺ T cells that upregulate multiple inhibitory receptors are not entirely inert and exhibit functional capacities. For example in chronic viral infection, subsets of PD-1⁺ exhausted CD8⁺ T cells have been found. The cell populations described herein provide a method of identifying, tracking and isolating these cell populations.

Targeted immunotherapies to counteract the mechanisms of tumor-induced T cell dysfunction have successfully provided persistent clinical benefits to patients. Most recently, these immunotherapies have focused on immune checkpoint blockade with anti-CTLA-4 and/or PD-1 mAbs, which are beneficial to a growing number of solid and hematological tumors and can be applicable to chronic infection. Blockade of immune checkpoints can also be combined with CD47 blockade and other therapies, including administration of the cognate antigen for the T cells. For example, checkpoint blockade may be combined with (a) blocking mAbs targeting additional inhibitory receptors, e.g. CTLA-4, LAG-3, TIM3, etc.; (b) inhibitors of soluble mediators targeting IDO, A2aR, CSF1R, IL-10 or TGFβ; (c) agonistic mAbs targeting activating receptors on T cells, e.g. CD137, OX40, GITR, or APCs, e.g. anti-CD40 mAbs; (d) vaccines or intratumoral injections of antigen to prime and/or expand antigen specific cells; (f) adoptive transfer of tumor infiltrating lymphocytes or chimeric antigen receptor (CAR) T cells; (g) Treg depletion; and the like.

SIRPα1 (PTPNS1, SHPS1), is a transmembrane glycoprotein, expressed primarily on myeloid and neuronal cells. SIRPα interacts with the widely distributed membrane protein CD47. In humans, the SIRPα protein is found in two major forms. One form, the variant 1 or V1 form, has the amino acid sequence set out as NCBI RefSeq NP_542970.1 (SEQ ID NO: 1) (residues 27-504 constitute the mature form). Another form, the variant 2 or V2 form, differs by 13 amino acids and has the amino acid sequence set out in GenBank as CAA71403.1 (residues 30-504 constitute the mature form). These two forms of SIRPα constitute about 80% of the forms of SIRPα present in humans, and both are embraced herein by the term “human SIRPα”. Also embraced by the term “human SIRPα” are the minor forms thereof that are endogenous to humans and have the same property of triggering signal transduction through CD47 upon binding thereto. Sequences of human SIRPα variants may be accessed through public databases, including Genbank accession numbers: ref|NP_542970.1; gb|EAX10606.1 (SEQ ID NO: 2); ref|XP_005260726.1 (SEQ ID NO: 3); gb|EAX10606.1 (SEQ ID NO: 4); XP_005260726.1 (SEQ ID NO: 5); gb|EAX10611.1 (SEQ ID NO: 6); gb|EAX10609.1 (SEQ ID NO: 7); dbj|BAA12974.1 (SEQ ID NO: 8); gb|AAH26692.1 (SEQ ID NO: 9); ref|XP_011527475.1 (SEQ ID NO: 10). See, for example Lee et al. (2007) J. Immunol. 179(11):7741-7750; herein specifically incorporated by reference.

Biological sample. The term “sample” with respect to an individual encompasses blood and other liquid samples of biological origin, solid tissue samples such as a biopsy specimen or tissue cultures or cells derived or isolated therefrom and the progeny thereof. The definition also includes samples that have been manipulated in any way after their procurement, such as by treatment with reagents; washed; or enrichment for certain cell populations, such as cancer cells. The definition also includes samples that have been enriched for particular types of molecules, e.g., nucleic acids, polypeptides, etc.

The term “sample” with respect to a patient encompasses blood and other liquid samples of biological origin, solid tissue samples such as a biopsy specimen or tissue cultures or cells derived therefrom and the progeny thereof. The definition also includes samples that have been manipulated in any way after their procurement, such as by treatment with reagents; washed; or enrichment for certain cell populations, such as cancer cells. The definition also includes sample that have been enriched for particular types of molecules, e.g., nucleic acids, polypeptides, etc.

The term “biological sample” encompasses a clinical sample, and also includes tissue obtained by surgical resection, tissue obtained by biopsy, cells in culture, cell supernatants, cell lysates, tissue samples, organs, bone marrow, blood, plasma, serum, and the like. A “biological sample” includes a sample obtained from a patient's infected cell, e.g., a sample comprising polynucleotides and/or polypeptides that is obtained from a patient's infected cell (e.g., a cell lysate or other cell extract comprising polynucleotides and/or polypeptides); and a sample comprising infected cells from a patient. A biological sample comprising an infected cell from a patient can also include non-infected cells.

Obtaining and assaying a sample. The term “assaying” is used herein to include the physical steps of manipulating a biological sample to generate data related to the sample. As will be readily understood by one of ordinary skill in the art, a biological sample must be “obtained” prior to assaying the sample. Thus, the term “assaying” implies that the sample has been obtained. The terms “obtained” or “obtaining” as used herein encompass the act of receiving an extracted or isolated biological sample. For example, a testing facility can “obtain” a biological sample in the mail (or via delivery, etc.) prior to assaying the sample. In some such cases, the biological sample was “extracted” or “isolated” from an individual by another party prior to mailing (i.e., delivery, transfer, etc.), and then “obtained” by the testing facility upon arrival of the sample. Thus, a testing facility can obtain the sample and then assay the sample, thereby producing data related to the sample.

The terms “obtained” or “obtaining” as used herein can also include the physical extraction or isolation of a biological sample from a subject. Accordingly, a biological sample can be isolated from a subject (and thus “obtained”) by the same person or same entity that subsequently assays the sample. When a biological sample is “extracted” or “isolated” from a first party or entity and then transferred (e.g., delivered, mailed, etc.) to a second party, the sample was “obtained” by the first party (and also “isolated” by the first party), and then subsequently “obtained” (but not “isolated”) by the second party. Accordingly, in some embodiments, the step of obtaining does not comprise the step of isolating a biological sample.

In some embodiments, the step of obtaining comprises the step of isolating a biological sample (e.g., a pre-treatment biological sample, a post-treatment biological sample, etc.). Methods and protocols for isolating various biological samples (e.g., a blood sample, a serum sample, a plasma sample, a biopsy sample, an aspirate, etc.) will be known to one of ordinary skill in the art and any convenient method may be used to isolate a biological sample.

The terms “determining”, “measuring”, “evaluating”, “assessing,” “assaying,” and “analyzing” are used interchangeably herein to refer to any form of measurement, and include determining if an element is present or not. These terms include both quantitative and/or qualitative determinations. Assaying may be relative or absolute. For example, “assaying” can be determining whether the expression level is less than or “greater than or equal to” a particular threshold, (the threshold can be pre-determined or can be determined by assaying a control sample). On the other hand, “assaying to determine the expression level” can mean determining a quantitative value (using any convenient metric) that represents the level of expression (i.e., expression level, e.g., the amount of protein and/or RNA, e.g., mRNA).

Immune Responsiveness Modulators. Immune checkpoint proteins are immune inhibitory molecules that act to decrease immune responsiveness of T cells toward a target cell, particularly against a chronically infected cell or a tumor cell. Responses to tumors and chronically infected cells by T cells can be dysregulated by tumor cells activating immune checkpoints (immune inhibitory proteins) and inhibiting co-stimulatory receptors (immune activating proteins). The class of therapeutic agents referred to in the art as “immune checkpoint inhibitors” reverses the inhibition of immune responses through administering antagonists of inhibitory signals. In some embodiments, an analysis for the presence of functional CD8⁺, SIRPα⁺ functional T cells is used to determine if patient comprises sufficient functional T cells for treatment, to determine if there has been an expansion of functional T cells following treatment, to isolate functional T cells for in vitro activation and expansion, to determine suitable specific combinations of agents and dosing schedules, and the like.

The immune-checkpoint receptors that have been most actively studied in the context of clinical cancer immunotherapy, cytotoxic T-lymphocyte-associated antigen 4 (CTLA4; also known as CD152) and programmed cell death protein 1 (PD1; also known as CD279)—are both inhibitory receptors. The clinical activity of antibodies that block either of these receptors shows that immunity can be enhanced at multiple levels and that combinatorial strategies can be intelligently designed, guided by mechanistic considerations and preclinical models.

Immune-checkpoint proteins of interest include PD-1 and PD-L1. Antibodies in current clinical use against these targets include nivolumab and pembrolizumab. The major role of PD1 is to limit the activity of T cells in peripheral tissues at the time of an inflammatory response to infection and to limit autoimmunity. PD1 expression is induced when T cells become activated. When engaged by one of its ligands, PD1 inhibits kinases that are involved in T cell activation. The two ligands for PD1 are PD1 ligand 1 (PDL1; also known as B7-H1 and CD274) and PDL2 (also known as B7-DC and CD273). The PD1 ligands are commonly upregulated on the tumor cell surface from many different human tumors. On cells from solid tumors, the major PD1 ligand that is expressed is PDL1. PDL1 is expressed on cancer cells and through binding to its receptor PD1 on T cells it inhibits T cell activation/function. Therefore, PD1 and PDL1 blocking agents can overcome this inhibitory signaling and maintain or restore anti-tumor T cell or anti-infectious function.

PDL1 is expressed on cancer cells and through binding to its receptor PD1 on T cells it inhibits T cell activation/function. Therefore, PD1 and PDL1 blocking agents can overcome this inhibitory signaling and maintain or restore anti-tumor T cell function. However, since PDL1 is expressed on tumor cells, antibodies that bind and block PDL1 can also enable ADCP, ADCC, and CDC of tumor cells.

CTLA4 is expressed exclusively on T cells where it primarily regulates the amplitude of the early stages of T cell activation. CTLA4 counteracts the activity of the T cell co-stimulatory receptor, CD28. CD28 and CTLA4 share identical ligands: CD80 (also known as B7.1) and CD86 (also known as B7.2). CTLA4 blockade results in a broad enhancement of immune responses. Two fully humanized CTLA4 antibodies, ipilimumab and tremelimumab, are in clinical testing and use. Clinically the response to immune-checkpoint blockers is slow and, in many patients, delayed up to 6 months after treatment initiation. In some cases, metastatic lesions actually increase in size on computed tomography (CT) or magnetic resonance imaging (MRI) scans before regressing. Anti-CTLA4 antibodies that antagonize this inhibitory immune function are very potent therapeutics but have significant side effects since this enables also T cell activity against the self that is usually inhibited through these inhibitory molecules and pathways. A combination with an agent that blockades CD47 activity may be beneficial.

Lymphocyte activation gene 3 (LAG3; also known as CD223), 2B4 (also known as CD244), B and T lymphocyte attenuator (BTLA; also known as CD272), T cell membrane protein 3 (TIM3; also known as HAVcr2), adenosine A2a receptor (A2aR) and the family of killer inhibitory receptors have each been associated with the inhibition of lymphocyte activity and in some cases the induction of T cell exhaustion. Antibody targeting of these receptors can be used in the methods of the invention.

LAG3 is a CD4 homolog that enhances the function of T_{Reg} cells. LAG3 also inhibits CD8⁺ effector T cell functions independently of its role on T_{Reg} cells. The only known ligand for LAG3 is MHC class II molecules, which are expressed on tumor-infiltrating macrophages and dendritic cells. LAG3 is one of various immune-checkpoint receptors that are coordinately upregulated on both T_{Reg} cells and anergic T cells, and simultaneous blockade of these receptors can result in enhanced reversal of this anergic state relative to blockade of one receptor alone. In particular, PD1 and LAG3 are commonly co-expressed on anergic or exhausted T cells. Dual blockade of LAG3 and PD1 synergistically reversed anergy among tumor-specific CD8⁺ T cells and virus-specific CD8⁺ T cells in the setting of chronic infection. LAG3 blocking agents can overcome this inhibitory signaling and maintain or restore anti-tumor T cell function.

TIM3 inhibits T helper 1 (T_{H}1) cell responses, and TIM3 antibodies enhance antitumor immunity. TIM3 has also been reported to be co-expressed with PD1 on tumor-specific CD8⁺ T cells. Tim3 blocking agents can overcome this inhibitory signaling and maintain or restore anti-tumor T cell function.

As used herein, the term “infection” refers to any state in at least one cell of an organism (i.e., a subject) is infected by an infectious agent (e.g., a subject has an intracellular pathogen infection, e.g., a chronic intracellular pathogen infection). As used herein, the term “infectious agent” refers to a foreign biological entity (i.e. a pathogen) that induces increased CD47 expression or upregulation of pro-phagocytic “eat me” signals in at least one cell of the infected organism. For example, infectious agents include, but are not limited to bacteria, viruses, protozoans, and fungi. Intracellular pathogens are of particular interest. Infectious diseases are disorders caused by infectious agents. Some infectious agents cause no recognizable symptoms or disease under certain conditions, but have the potential to cause symptoms or disease under changed conditions. The subject methods can be used in the treatment of chronic pathogen infections, for example including but not limited to viral infections, e.g. retrovirus, lentivirus, hepadna virus, herpes viruses, pox viruses, human papilloma viruses, etc.; intracellular bacterial infections, e.g. Mycobacterium, Chlamydophila, Ehrlichia, Rickettsia, Brucella, Legionella, Francisella, Listeria, Coxiella, Neisseria, Salmonella, Yersinia sp, Helicobacter pylori etc.; and intracellular protozoan pathogens, e.g. Plasmodium sp, Trypanosoma sp., Giardia sp., Toxoplasma sp., Leishmania sp., etc.

The terms “recipient”, “individual”, “subject”, “host”, and “patient”, are used interchangeably herein and refer to any mammalian subject for whom diagnosis, treatment, or therapy is desired, particularly humans. “Mammal” for purposes of treatment refers to any animal classified as a mammal, including humans, domestic and farm animals, and zoo, sports, or pet animals, such as dogs, horses, cats, cows, sheep, goats, pigs, etc. Preferably, the mammal is human.

A “therapeutically effective dose” or “therapeutic dose” is an amount sufficient to effect desired clinical results (i.e., achieve therapeutic efficacy). A therapeutically effective dose can be administered in one or more administrations. For purposes of this invention, a therapeutically effective dose of an agent is an amount that is sufficient to palliate, ameliorate, stabilize, reverse, prevent, slow or delay the progression of the disease state (e.g., cancer or chronic infection) by increasing activity of CD8⁺ T cells.

Anti-CD47 agent. As used herein, the term “anti-CD47 agent” or “CD47-blocking agent” refers to any agent that reduces the binding of CD47 (e.g., on a target cell) to SIRPα (e.g., on a phagocytic cell). Non-limiting examples of suitable anti-CD47 reagents include SIRPα polypeptides, e.g. high affinity SIRPα polypeptides; anti-SIRPα antibodies; soluble CD47 polypeptides; and anti-CD47 antibodies or antibody fragments; and conjugates thereof, e.g. soluble SIRPα polypeptides conjugated to an Fc region polypeptide. In some embodiments, a suitable anti-CD47 agent specifically binds CD47 to reduce the binding of CD47 to SIRPα.

In some embodiments, a suitable anti-CD47 agent, e.g., an anti-SIRPα antibody, a soluble CD47 polypeptide, etc., specifically binds to SIRPα to reduce the binding of CD47 to SIRPα. A suitable anti-CD47 agent that binds SIRPα does not activate SIRPα (e.g., in the SIRPα-expressing phagocytic cell). The efficacy of a suitable anti-CD47 agent can be assessed by assaying the agent (further described below). In an exemplary assay, target cells are incubated in the presence or absence of the candidate agent. An agent for use in the methods of the invention will up-regulate phagocytosis by at least 5% (e.g., at least 10%, at least 20%, at least 30%, at least 40%, at least 50%, at least 60%, at least 70%, at least 80%, at least 90%, at least 100%, at least 120%, at least 140%, at least 160%, at least 180%, at least 200%, at least 500%, at least 1000%) compared to phagocytosis in the absence of the agent. Similarly, an in vitro assay for levels of tyrosine phosphorylation of SIRPα will show a decrease in phosphorylation by at least 5% (e.g., at least 10%, at least 15%, at least 20%, at least 30%, at least 40%, at least 50%, at least 60%, at least 70%, at least 80%, at least 90%, or 100%) compared to phosphorylation observed in absence of the candidate agent.

In some embodiments, the anti-CD47 agent does not activate CD47 upon binding. When CD47 is activated, a process akin to apoptosis (i.e., programmed cell death) may occur (Manna and Frazier (2004) Cancer Research, 64, 1026-1036). Thus, in some embodiments, the anti-CD47 agent does not directly induce cell death of a CD47-expressing cell.

SIRPα polypeptide. A SIRPα polypeptide comprises the portion of SIRPα that is sufficient to bind CD47 at a recognizable affinity, which portion normally lies between the signal sequence and the transmembrane domain, or a fragment thereof that retains the binding activity. A suitable SIRPα polypeptide reduces (e.g., blocks, prevents, etc.) the interaction between the native proteins SIRPα and CD47. The SIRPα reagent will usually comprise at least the dl domain of SIRPα. In some embodiments, a SIRPα reagent is a fusion protein, e.g., fused in frame with a second polypeptide. In some embodiments, the second polypeptide is capable of increasing the size of the fusion protein, e.g., so that the fusion protein will not be cleared from the circulation rapidly. In some embodiments, the second polypeptide is part or whole of an immunoglobulin Fc region. The Fc region aids in phagocytosis by providing an “eat me” signal, which enhances the block of the “don't eat me” signal provided by the high affinity SIRPα reagent. In other embodiments, the second polypeptide is any suitable polypeptide that is substantially similar to Fc, e.g., providing increased size, multimerization domains, and/or additional binding or interaction with Ig molecules.

Included as a SIRPα polypeptide are high-affinity variants of SIRPα as known and used in the art, including without limitation CV1-hIgG4, which has the set of amino acid substitutions relative to wild-type SIRPα of V61; V271; I31 F; E47V; K53R; E54Q; H56P; S66T; V92I and is fused to an Fc region. High affinity SIRPα reagents are described in international application PCT/US13/21937, which is hereby specifically incorporated by reference. In some embodiments, a high affinity SIRPα reagent is soluble, where the polypeptide lacks the SIRPα transmembrane domain and comprises at least one amino acid change relative to the wild-type SIRPα sequence, and wherein the amino acid change increases the affinity of the SIRPα polypeptide binding to CD47, for example by decreasing the off-rate by at least 10-fold, at least 20-fold, at least 50-fold, at least 100-fold, at least 500-fold, or more. The high affinity SIRPα reagent will usually comprise at least the dl domain of SIRPα with modified amino acid residues to increase affinity. The amino acid changes that provide for increased affinity are localized in the dl domain, and thus high affinity SIRPα reagents comprise a dl domain of human SIRPα, with at least one amino acid change relative to the wild-type sequence within the dl domain. Such a high affinity SIRPα reagent optionally comprises additional amino acid sequences, for example antibody Fc sequences; portions of the wild-type human SIRPα protein other than the dl domain, including without limitation residues 150 to 374 of the native protein or fragments thereof, usually fragments contiguous with the dl domain; and the like. High affinity SIRPα reagents may be monomeric or multimeric, i.e. dimer, trimer, tetramer, etc.

Anti-CD47 antibodies. In some embodiments, a subject anti-CD47 agent is an antibody that specifically binds CD47 (i.e., an anti-CD47 antibody) and reduces the interaction between CD47 on one cell (e.g., an infected cell) and SIRPα on another cell (e.g., a phagocytic cell). In some embodiments, a suitable anti-CD47 antibody does not activate CD47 upon binding. Some anti-CD47 antibodies do not reduce the binding of CD47 to SIRPα (and are therefore not considered to be an “anti-CD47 agent” herein) and such an antibody can be referred to as a “non-blocking anti-CD47 antibody.” A suitable anti-CD47 antibody that is an “anti-CD47 agent” can be referred to as a “CD47-blocking antibody”. A non-limiting example of a non-blocking antibody is anti-CD47 antibody 2D3, which binds to CD47, but does not reduce the interaction between CD47 and SIRPα. Non-limiting examples of suitable antibodies include clones B6H12, 5F9, 8B6, and C3 (for example as described in International Patent Publication WO 2011/143624, herein specifically incorporated by reference). Suitable anti-CD47 antibodies include fully human, humanized or chimeric versions of such antibodies. Humanized antibodies (e.g., hu5F9-G4) are especially useful for in vivo applications in humans due to their low antigenicity. Similarly caninized, felinized, etc. antibodies are especially useful for applications in dogs, cats, and other species respectively. Antibodies of interest include humanized antibodies, or caninized, felinized, equinized, bovinized, porcinized, etc., antibodies, and variants thereof.

Anti-SIRPα antibodies. Antibodies that specifically bind to human SIRPα are known and used in the art, and may be adapted by the use of an engineered Fc region. Exemplary antibodies include those described in international patent application WO 2015/138600; in published US application 2014/0242095 (University Health Networks); published application CN103665165 (JIANGSU KUANGYA BIOLOGICAL MEDICAL SCIENCE & TECHNOLOGY; Zhao XW et al. Proc Natl Acad Sci USA 108:18342-7 (2011), each herein specifically incorporated by reference. An anti-SIRPα antibody may be pan-specific, i.e. binding to two or more different human SIRPα isoforms; or may be specific for one isoform. For example, the antibody 1.23A described by Zhang et al., supra. is reported to be specific for the SIRPα1 variant, while the 12C4 antibody is pan-specific. Anti-SIRPα antibodies can also be specific for SIRPα and lack binding to SIRPβ and/or SIRPγ. Anti-SIRPα antibodies can be pan-specific with respect to SIRPβ and/or SIRPγ.

Suitable anti-SIRPα antibodies can bind SIRPα without activating or stimulating signaling through SIRPα because activation of SIRPα would inhibit phagocytosis. Instead, suitable anti-SIRPα antibodies facilitate the preferential phagocytosis of inflicted cells over normal cells. Those cells that express higher levels of CD47 (e.g., infected cells) relative to other cells (non-infected cells) will be preferentially phagocytosed. Thus, a suitable anti-SIRPα antibody specifically binds SIRPα (without activating/stimulating enough of a signaling response to inhibit phagocytosis) and blocks an interaction between SIRPα and CD47. Suitable anti-SIRPα antibodies include fully human, humanized or chimeric versions of such antibodies. Humanized antibodies are especially useful for in vivo applications in humans due to their low antigenicity. Similarly caninized, felinized, etc. antibodies are especially useful for applications in dogs, cats, and other species respectively. Antibodies of interest include humanized antibodies, or caninized, felinized, equinized, bovinized, porcinized, etc., antibodies, and variants thereof.

Soluble CD47 polypeptides. In some embodiments, a subject anti-CD47 agent is a soluble CD47 polypeptide that specifically binds SIRPα and reduces the interaction between CD47 on one cell (e.g., an infected cell) and SIRPα on another cell (e.g., a phagocytic cell). A suitable soluble CD47 polypeptide can bind SIRPα without activating or stimulating signaling through SIRPα because activation of SIRPα would inhibit phagocytosis. Instead, suitable soluble CD47 polypeptides facilitate the preferential phagocytosis of infected cells over non-infected cells. Those cells that express higher levels of CD47 (e.g., infected cells) relative to normal, non-target cells (normal cells) will be preferentially phagocytosed. Thus, a suitable soluble CD47 polypeptide specifically binds SIRPα without activating/stimulating enough of a signaling response to inhibit phagocytosis. In some cases, a suitable soluble CD47 polypeptide can be a fusion protein (for example as structurally described in US Patent Publication US20100239579, herein specifically incorporated by reference). However, only fusion proteins that do not activate/stimulate SIRPα are suitable for the methods provided herein. Suitable soluble CD47 polypeptides also include any peptide or peptide fragment comprising variant or naturally existing CD47 sequences (e.g., extracellular domain sequences or extracellular domain variants) that can specifically bind SIRPα and inhibit the interaction between CD47 and SIRPα without stimulating enough SIRPα activity to inhibit phagocytosis.

In certain embodiments, soluble CD47 polypeptide comprises the extracellular domain of CD47, including the signal peptide, such that the extracellular portion of CD47 is typically 142 amino acids in length, and has the amino acid sequence set forth in, for example, the Genbank reference sequence for human CD47, including NP_942088 or NP_001768.1. The soluble CD47 polypeptides described herein also include CD47 extracellular domain variants that comprise an amino acid sequence at least 65%-75%, 75%-80%, 80-85%, 85%-90%, or 95%-99% (or any percent identity not specifically enumerated between 65% to 100%), which variants retain the capability to bind to SIRPα without stimulating SIRPα signaling.

The terms “treatment”, “treating”, “treat” and the like are used herein to generally refer to obtaining a desired pharmacologic and/or physiologic effect. The effect can be prophylactic in terms of completely or partially preventing a disease or symptom(s) thereof and/or may be therapeutic in terms of a partial or complete stabilization or cure for a disease and/or adverse effect attributable to the disease. The term “treatment” encompasses any treatment of a disease in a mammal, particularly a human, and includes: (a) preventing the disease and/or symptom(s) from occurring in a subject who may be predisposed to the disease or symptom but has not yet been diagnosed as having it; (b) inhibiting the disease and/or symptom(s), i.e., arresting their development; or (c) relieving the disease symptom(s), i.e., causing regression of the disease and/or symptom(s). Those in need of treatment include those already inflicted (e.g., those with cancer, those with an infection, etc.) as well as those in which prevention is desired (e.g., those with increased susceptibility to cancer, those suspected of having cancer, etc.).

A target cell can have cancer, can harbor an infection (e.g., a chronic infection), and other hyper-proliferative conditions, for example sclerosis, fibrosis, and the like, etc. that result in T cell exhaustion. “Inflicted cells” may be those cells that cause the symptoms, illness, or disease. As non-limiting examples, the inflicted cells of a patient can be cancer cells, infected cells, and the like.

A therapeutic treatment is one in which the subject is inflicted prior to administration and a prophylactic treatment is one in which the subject is not inflicted prior to administration. In some embodiments, the subject has an increased likelihood of becoming inflicted or is suspected of being inflicted prior to treatment. In some embodiments, the subject is suspected of having an increased likelihood of becoming inflicted.

Examples of symptoms, illnesses, and/or diseases that can be treated with the methods described herein include, but are not limited to cancer and infection (e.g., chronic infection). As used herein “cancer” includes any form of cancer (e.g., leukemia; acute myeloid leukemia (AML); acute lymphoblastic leukemia (ALL); metastasis; minimal residual disease; solid tumor cancers, e.g., lung, prostate, breast, bladder, colon, ovarian, glioblastoma, medulloblastoma, leiomyosarcoma, and head & neck squamous cell carcinomas, melanomas; etc.).

The terms “specific binding,” “specifically binds,” and the like, refer to non-covalent or covalent preferential binding to a molecule relative to other molecules or moieties in a solution or reaction mixture (e.g., an antibody specifically binds to a particular polypeptide or epitope relative to other available polypeptides, or binding of a SIRPα polypeptide). In some embodiments, the affinity of one molecule for another molecule to which it specifically binds is characterized by a K_{D} (dissociation constant) of 10⁻⁵ M or less (e.g., 10⁻⁶ M or less, 10⁻⁷ M or less, 10⁻⁸ M or less, 10⁻⁹ M or less, 10⁻¹⁰ M or less, 10⁻¹¹ M or less, 10⁻¹² M or less, 10⁻¹³ M or less, 10⁻¹⁴ M or less, 10⁻¹⁵ M or less, or 10⁻¹⁶ M or less). “Affinity” refers to the strength of binding, increased binding affinity being correlated with a lower K_{D}.

The term “specific binding member” as used herein refers to a member of a specific binding pair (i.e., two molecules, usually two different molecules, where one of the molecules, e.g., a first specific binding member, through non-covalent means specifically binds to the other molecule, e.g., a second specific binding member). Suitable specific binding members include agents that specifically bind CD47 and/or SIRPα (i.e., anti-CD47 agents), or that otherwise block the interaction between CD47 and SIRPα.

The term “antibody” is used in the broadest sense and specifically covers monoclonal antibodies (including full length monoclonal antibodies), polyclonal antibodies, multispecific antibodies (e.g., bispecific antibodies), and antibody fragments so long as they exhibit the desired biological activity. “Antibodies” (Abs) and “immunoglobulins” (Igs) are glycoproteins having the same structural characteristics. While antibodies exhibit binding specificity to a specific antigen, immunoglobulins include both antibodies and other antibody-like molecules which lack antigen specificity. Polypeptides of the latter kind are, for example, produced at low levels by the lymph system and at increased levels by myelomas.

“Antibody fragment”, and all grammatical variants thereof, as used herein are defined as a portion of an intact antibody comprising the antigen binding site or variable region of the intact antibody, wherein the portion is free of the constant heavy chain domains (i.e. CH2, CH3, and CH4, depending on antibody isotype) of the Fc region of the intact antibody. Examples of antibody fragments include Fab, Fab′, Fab′-SH, F(ab′)₂, and Fv fragments; diabodies; any antibody fragment that is a polypeptide having a primary structure consisting of one uninterrupted sequence of contiguous amino acid residues (referred to herein as a “single-chain antibody fragment” or “single chain polypeptide”), including without limitation (1) single-chain Fv (scFv) molecules (2) single chain polypeptides containing only one light chain variable domain, or a fragment thereof that contains the three CDRs of the light chain variable domain, without an associated heavy chain moiety (3) single chain polypeptides containing only one heavy chain variable region, or a fragment thereof containing the three CDRs of the heavy chain variable region, without an associated light chain moiety and (4) nanobodies comprising single Ig domains from non-human species or other specific single-domain binding modules; and multispecific or multivalent structures formed from antibody fragments. In an antibody fragment comprising one or more heavy chains, the heavy chain(s) can contain any constant domain sequence (e.g. CH1 in the IgG isotype) found in a non-Fc region of an intact antibody, and/or can contain any hinge region sequence found in an intact antibody, and/or can contain a leucine zipper sequence fused to or situated in the hinge region sequence or the constant domain sequence of the heavy chain(s).

As used in this invention, the term “epitope” means any antigenic determinant on an antigen to which the paratope of an antibody binds. Epitopic determinants usually consist of chemically active surface groupings of molecules such as amino acids or sugar side chains and usually have specific three dimensional structural characteristics, as well as specific charge characteristics.

“Providing an analysis” is used herein to refer to the delivery of an oral or written analysis (i.e., a document, a report, etc.). A written analysis can be a printed or electronic document. A suitable analysis (e.g., an oral or written report) provides any or all of the following information: identifying information of the subject (name, age, etc.), a description of what type of biological sample(s) was used and/or how it was used, the technique used to assay the sample, the results of the assay, the assessment as to whether the individual is determined to be responsive or not responsive to the anti-CD47 agent, a recommendation to continue or alter therapy, a recommended strategy for additional therapy, etc. The report can be in any format including, but not limited to printed information on a suitable medium or substrate (e.g., paper); or electronic format. If in electronic format, the report can be in any computer readable medium, e.g., diskette, compact disk (CD), flash drive, and the like, on which the information has been recorded. In addition, the report may be present as a website address which may be used via the internet to access the information at a remote site.

Methods

Methods are provided for determining whether an individual has functional T cells, e.g. in a population of T cells bearing markers of T cell exhaustion, e.g. PD-1⁺ CD8⁺ T cells. Prolonged exposure of CD8⁺ T cells to antigenic stimulation leads to a state of diminished function, termed exhaustion. It is shown herein that during exhaustion there is a subset of functional CD8⁺ T cells defined by surface expression of SIRPα protein. On SIRP+ CD8⁺ T cells, expression of coinhibitory receptors is counterbalanced by expression of co-stimulatory receptors and it is only these SIRP+ cells that actively proliferate, transcribe IFNγ and show cytolytic activity. SIRP+ CD8⁺ T cells are present in patients with chronic infections. Therapeutic blockade of PD-L1 or other inhibitory receptors to reinvigorate CD8⁺ T cells during chronic infection expands the cytotoxic subset of SIRP+CD8⁺ T cells.

In some embodiments, methods are provided for identifying functional CD8⁺ T cells by cell surface expression of SIRPα. In some embodiments, the functional CD8+ cells are identifying in a population of exhausted T cells. In some embodiments the T cells are positive for one or more inhibitory receptors, which include, without limitation, PD-1, CTLA-4, LAG-3, TIM-3, etc. In some embodiments the cells co-express PD-1 and SIRPα. In some embodiments the exhausted T cells are specific for a tumor antigen. In some embodiments the exhausted T cells are specific for an antigen of a pathogen in a chronic infection, which include, without limitation, viral and bacterial antigens.

It will be understood by one of ordinary skill in the art that in some cases, it is convenient to wait until multiple samples (e.g., a pre-treatment biological sample and a post-treatment biological sample) have been obtained prior to assaying the samples. Accordingly, in some cases an isolated biological sample (e.g., a pre-treatment biological sample, a post-treatment biological sample, etc.) is stored until all appropriate samples have been obtained. One of ordinary skill in the art will understand how to appropriately store a variety of different types of biological samples and any convenient method of storage may be used (e.g., refrigeration) that is appropriate for the particular biological sample. In some embodiments, a pre-treatment biological sample is assayed prior to obtaining a post-treatment biological sample. In some cases, a pre-treatment biological sample and a post-treatment biological sample are assayed in parallel. In some cases, multiple different post-treatment biological samples and/or a pre-treatment biological sample are assayed in parallel. In some cases, biological samples are processed immediately or as soon as possible after they are obtained.

In subject methods, the concentration (i.e., “level”), or expression level of a gene product, which may be an RNA, a protein, etc., in a biological sample is measured (i.e., “determined”). Specifically, the expression of SIRPα by CD8⁺ T cells, which may be PD-1⁺ CD8⁺ T cells, is determined. By “expression level” (or “level”) it is meant the level of gene product (e.g. the absolute and/or normalized value determined for the RNA expression level of a biomarker or for the expression level of the encoded polypeptide, or the concentration of the protein in a biological sample). The term “gene product” or “expression product” are used herein to refer to the RNA transcription products (RNA transcripts, e.g. mRNA, an unspliced RNA, a splice variant mRNA, and/or a fragmented RNA) of the gene, including mRNA, and the polypeptide translation products of such RNA transcripts.

The terms “determining”, “measuring”, “evaluating”, “assessing,” “assaying,” and “analyzing” are used interchangeably herein to refer to any form of measurement, and include determining if an element is present or not. These terms include both quantitative and/or qualitative determinations. Assaying may be relative or absolute. For example, “assaying” can be determining whether the expression level is less than or “greater than or equal to” a particular threshold, (the threshold can be pre-determined or can be determined by assaying a control sample). On the other hand, “assaying to determine the expression level” can mean determining a quantitative value (using any convenient metric) that represents the level of expression (i.e., expression level, e.g., the amount of protein and/or RNA, e.g., mRNA) of a particular biomarker. The level of expression can be expressed in arbitrary units associated with a particular assay (e.g., fluorescence units, e.g., mean fluorescence intensity (MFI)), or can be expressed as an absolute value with defined units (e.g., number of mRNA transcripts, number of protein molecules, concentration of protein, etc.). Additionally, the level of expression of a biomarker can be compared to the expression level of one or more additional genes (e.g., nucleic acids and/or their encoded proteins) to derive a normalized value that represents a normalized expression level. The specific metric (or units) chosen is not crucial as long as the same units are used (or conversion to the same units is performed) when evaluating multiple biological samples from the same individual (e.g., biological samples taken at different points in time from the same individual). This is because the units cancel when calculating a fold-change (i.e., determining a ratio) in the expression level from one biological sample to the next (e.g., biological samples taken at different points in time from the same individual).

For measuring RNA levels, the amount or level of an RNA in the sample is determined, e.g., the level of an mRNA. In some instances, the expression level of one or more additional RNAs may also be measured, and the level of biomarker expression compared to the level of the one or more additional RNAs to provide a normalized value for expression level. Any convenient protocol for evaluating RNA levels may be employed wherein the level of one or more RNAs in the assayed sample is determined.

A number of exemplary methods for measuring RNA (e.g., mRNA) expression levels (e.g., expression level of a nucleic acid biomarker) in a sample are known by one of ordinary skill in the art, and any convenient method can be used. Exemplary methods include, but are not limited to: hybridization-based methods (e.g., Northern blotting, array hybridization (e.g., microarray); in situ hybridization; in situ hybridization followed by FACS; and the like)(Parker & Barnes, Methods in Molecular Biology 106:247-283 (1999)); RNAse protection assays (Hod, Biotechniques 13:852-854 (1992)); PCR-based methods (e.g., reverse transcription PCR (RT-PCR), quantitative RT-PCR (qRT-PCR), real-time RT-PCR, etc.)(Weis et al., Trends in Genetics 8:263-264 (1992)); nucleic acid sequencing methods (e.g., Sanger sequencing, Next Generation sequencing (i.e., massive parallel high throughput sequencing, e.g., Illumina's reversible terminator method, Roche's pyrosequencing method (454), Life Technologies' sequencing by ligation (the SOLiD platform), Life Technologies' Ion Torrent platform, single molecule sequencing, etc.); and the like.

In some embodiments, the biological sample can be assayed directly. In some embodiments, nucleic acid of the biological sample is amplified (e.g., by PCR) prior to assaying. As such, techniques such as PCR (Polymerase Chain Reaction), RT-PCR (reverse transcriptase PCR), qRT-PCR (quantitative RT-PCR, real time RT-PCR), etc. can be used prior to the hybridization methods and/or the sequencing methods discussed above.

For measuring protein levels, particularly protein levels present on a T cell surface, the amount or level of a polypeptide in the biological sample is determined. In some embodiments, the extracellular protein level is measured. In some embodiments concentration is a relative value measured by comparing the level of one protein relative to another protein. In other embodiments the concentration is an absolute measurement of weight/volume or weight/weight.

In some cases, the cells are removed from the biological sample (e.g., via centrifugation, via adhering cells to a dish or to plastic, etc.) prior to measuring the concentration. In some instances, the concentration of one or more additional proteins may also be measured, and SIRPα concentration compared to the level of the one or more additional proteins to provide a normalized value for the SIRPα concentration. Any convenient protocol for evaluating protein levels may be employed wherein the level of one or more proteins in the assayed sample is determined.

Clinical samples for use in the methods of the invention may be obtained from a variety of sources, particularly blood, although in some instances samples such as bone marrow, lymph, cerebrospinal fluid, synovial fluid, and the like may be used. Such samples can be separated by centrifugation, elutriation, density gradient separation, apheresis, affinity selection, panning, FACS, centrifugation with Hypaque, etc. prior to analysis, and usually a mononuclear fraction (PBMC) will be used. Once a sample is obtained, it can be used directly, frozen, or maintained in appropriate culture medium for short periods of time. Various media can be employed to maintain cells. The samples may be obtained by any convenient procedure, such as the drawing of blood, venipuncture, biopsy, or the like. Usually a sample will comprise at least about 10² cells, more usually at least about 10³ cells, and preferable 10⁴, 10⁵ or more cells. Typically the samples will be from human patients, although animal models may find use, e.g. equine, bovine, porcine, canine, feline, rodent, e.g. mice, rats, hamster, primate, etc.

An appropriate solution may be used for dispersion or suspension of the cell sample. Such solution will generally be a balanced salt solution, e.g. normal saline, PBS, Hank's balanced salt solution, etc., conveniently supplemented with fetal calf serum or other naturally occurring factors, in conjunction with an acceptable buffer at low concentration, generally from 5-25 mM. Convenient buffers include HEPES, phosphate buffers, lactate buffers, etc.

Analysis of the cell staining may use conventional methods. Techniques providing accurate enumeration include fluorescence activated cell sorters, which can have varying degrees of sophistication, such as multiple color channels, low angle and obtuse light scattering detecting channels, impedance channels, etc. The cells may be selected against dead cells by employing dyes associated with dead cells (e.g. propidium iodide).

The affinity reagents may be specific antibodies for the cell surface molecules indicated, e.g. CD8, PD-1, SIRPα, etc. Antibodies may be monoclonal or polyclonal, and may be produced by transgenic animals, immunized animals, immortalized human or animal B-cells, cells transfected with DNA vectors encoding the antibody, etc. The details of the preparation of antibodies and their suitability for use as specific binding members are well-known to those skilled in the art.

Of particular interest is the use of antibodies as affinity reagents. Conveniently, these antibodies are conjugated with a label for use in separation. Labels include magnetic beads, which allow for direct separation, biotin, which can be removed with avidin or streptavidin bound to a support, fluorochromes, which can be used with a fluorescence activated cell sorter, or the like, to allow for ease of separation of the particular cell type. Fluorochromes that find use include phycobiliproteins, e.g. phycoerythrin and allophycocyanins, fluorescein and Texas red. Frequently each antibody is labeled with a different fluorochrome, to permit independent sorting for each marker.

The antibodies are added to a suspension of cells, and incubated for a period of time sufficient to bind the available cell surface antigens. The incubation will usually be at least about 5 minutes and usually less than about 30 minutes. It is desirable to have a sufficient concentration of antibodies in the reaction mixture, such that the efficiency of the separation is not limited by lack of antibody. The appropriate concentration is determined by titration. The medium in which the cells are separated will be any medium that maintains the viability of the cells. A preferred medium is phosphate buffered saline containing from 0.1 to 0.5% BSA. Various media are commercially available and may be used according to the nature of the cells, including Dulbecco's Modified Eagle Medium (dMEM), Hank's Basic Salt Solution (HBSS), Dulbecco's phosphate buffered saline (dPBS), RPMI, Iscove's medium, PBS with 5 mM EDTA, etc., frequently supplemented with fetal calf serum, BSA, HSA, etc.

The labeled cells are then quantitated as to the expression of the cell surface markers as previously described.

The comparison of a differential analysis obtained from a patient sample, and a reference differential analysis; or of patient samples at varying time points, is accomplished by the use of suitable deduction protocols, AI systems, statistical comparisons, etc. An analysis of particular interest tracks a patient, e.g. prior to treatment with treatment with a checkpoint inhibitor, following treatment, etc. The methods of the invention provide detection of T cell expansion, and therefore allow therapeutic intervention to enhance treatment.

In some embodiments the infection is a chronic infection, i.e. an infection that is not cleared by the host immune system within a period of up to 1 week, 2 weeks, etc. In some cases, chronic infections involve integration of pathogen genetic elements into the host genome, e.g. retroviruses, lentiviruses, Hepatitis B virus, etc. In other cases, chronic infections, for example certain intracellular bacteria or protozoan pathogens, result from a pathogen cell residing within a host cell. Additionally, in some embodiments, the infection is in a latent stage, as with herpes viruses or human papilloma viruses.

Viral pathogens of interest include without limitation, retroviral and lentiviral pathogens, e.g. HIV-1; HIV-2, HTLV, FIV, SIV, etc., Hepatitis B virus, etc. Microbes of interest, but not limited to the following, include: Yersinia sp., e.g. Y. pestis, Y. pseudotuberculosis, Y enterocolitica; franciscella sp.; PastureIla sp.; Vibrio sp., e.g. V. cholerae, V. parahemolyticus; Legionella sp., e.g. L. pneumophila; Listeria sp., e.g. L. monocytogenes; Mycoplasma sp., e.g. M. hominis, M. pneumoniae; Mycobacterium sp., e.g. M. tuberculosis, M. leprae; Rickettsia sp., e.g. R. rickettsii, R. typhi; Chlamydia sp., e.g. C. trachomatis, C. pneumoniae, C. psittaci; Helicobacter sp., e.g. H. pylori, etc. Also included are intracellular protozoan pathogens, e.g. Plasmodium sp, Trypanosoma sp., Giardia sp., Toxoplasma sp., Leishmania sp., etc. In some cases, the pathogen is not a virus. In some cases, the pathogen is not a pox virus. In some cases, the pathogen is a virus, but is not a pox virus (i.e., the pathogen is a virus other than a pox virus). In some cases, the pathogen is a virus, but is not a vaccinia virus (i.e., the pathogen is a virus other than a vaccinia virus). In some cases, the pathogen is a virus, but is not a molluscum contagiosum virus (MCV) (i.e., the pathogen is a virus other than MCV). In some cases, the pathogen is a virus, but is not a pox virus, a vaccinia virus, or a molluscum contagiosum virus (MCV) (i.e., the pathogen is a virus other than a pox virus, vaccinia virus, or MCV). An infection treated with the methods of the invention generally involves a pathogen with at least a portion of its life-cycle within a host cell, i.e. an intracellular phase.

In some embodiments, the methods of the invention involve diagnosis of a patient as suffering from a pathogenic intracellular infection; or selection of a patient previously diagnosed as suffering from a pathogenic intracellular infection; treating the patient with a regimen to activate and expand functional CD8+SIRPα⁺ T cells, optionally in combination with an additional therapy; and monitoring the patient for efficacy of treatment. Monitoring may also include measure clinical indicia of infection, e.g. fever, white blood cell count, etc., and/or direct monitoring for presence of the pathogen.

Treatment of infection may be combined with other active agents. Classes of antibiotics include penicillins, e.g. penicillin G, penicillin V, methicillin, oxacillin, carbenicillin, nafcillin, ampicillin, etc.; penicillins in combination with β-lactamase inhibitors, cephalosporins, e.g. cefaclor, cefazolin, cefuroxime, moxalactam, etc.; carbapenems; monobactams; aminoglycosides; tetracyclines; macrolides; lincomycins; polymyxins; sulfonamides; quinolones; cloramphenical; metronidazole; spectinomycin; trimethoprim; vancomycin; etc. Cytokines may also be included, e.g. interferon γ, tumor necrosis factor α, interleukin 12, etc. Antiviral agents, e.g. acyclovir, gancyclovir, etc., may also be used in treatment.

The term “responsive” as used herein means that the therapeutic regimen is having the desired effect and the individual's body is responding appropriately to the administration by expansion, i.e. increased numbers, of functional CD8+SIRPα⁺ T cells. The term “continue treatment” (i.e., continue therapy) is used herein to mean that the current course of treatment is to continue. Alternatively, “altering therapy” is used herein to mean “discontinuing therapy” or “changing the therapy” (e.g., changing the particular dose and/or frequency of administration). In some cases, therapy can be altered, e.g., increased, until a dose and/or frequency is reached at which the individual is deemed to be responsive.

In some embodiments, the subject methods include providing an analysis indicating whether the individual is determined to be responsive or not responsive to therapy. As described above, an analysis can be an oral or written report (e.g., written or electronic document). The analysis can be provided to the subject, to the subject's physician, to a testing facility, etc. The analysis can also be accessible as a website address via the internet. In some such cases, the analysis can be accessible by multiple different entities (e.g., the subject, the subject's physician, a testing facility, etc.)

### Kits

Also provided are kits for use in the methods. The subject kits include a tool (e.g., a PCR primer pair specific for SIRPα, an antibody that specifically binds to SIRPα, and the like) for determining the level of expression. The subject kits can also include an immune checkpoint inhibitor, e.g. provided in a dosage form (e.g., a therapeutically effective dosage form).

In addition to the above components, the subject kits may further include (in certain embodiments) instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, and the like. Yet another form of these instructions is a computer readable medium, e.g., diskette, compact disk (CD), flash drive, and the like, on which the information has been recorded. Yet another form of these instructions that may be present is a website address which may be used via the internet to access the information at a removed site.

The invention now being fully described, it will be apparent to one of ordinary skill in the art that various changes and modifications can be made without departing from the spirit or scope of the invention.

### EXPERIMENTAL

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

All publications and patent applications cited in this specification are herein incorporated by reference as if each individual publication or patent application were specifically and individually indicated to be incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims.

### Example 1

### A Functional Subset of CD8⁺ T Cells During Chronic Exhaustion is Defined by SIRPα Expression

Prolonged exposure of CD8⁺ T cells to antigenic stimulation, as in chronic viral infections, leads to a state of diminished function termed exhaustion. We now demonstrate that even during exhaustion there is a subset of functional CD8⁺ T cells defined by surface expression of SIRPα, a protein not previously reported on lymphocytes. On SIRPα⁺ CD8⁺ T cells, expression of co-inhibitory receptors is counterbalanced by expression of co-stimulatory receptors and it is only SIRPα⁺ cells that actively proliferate, transcribe IFNγ and show cytolytic activity. Furthermore, target cells that express the ligand for SIRPα, CD47, are more susceptible to CD8⁺ T cell-killing in vivo. SIRPα⁺ CD8⁺ T cells are evident in mice infected with Friend retrovirus, LCMV Clone 13, and in patients with chronic HCV infections. Furthermore, therapeutic blockade of PD-L1 to reinvigorate CD8⁺ T cells during chronic infection expands the cytotoxic subset of SIRPα⁺ CD8⁺ T cells.

In this study we examine the expression of a novel cell surface maker, signal-regulatory protein alpha (SIRPα), expressed on exhausted CD8⁺ T cells during chronic infection of mice with Friend virus (FV), a naturally occurring retrovirus of mice. Like other chronic viral infections, chronic FV is associated with exhausted CD8⁺ T cells because of sustained antigenic stimulation and suppression by regulatory T cells. To identify cell surface markers that might be useful for the identification and therapeutic targeting of unique CD8⁺ T cell subsets, we analyzed a publicly available microarray database from CD8⁺ T cells isolated from mice chronically infected with LCMV Clone 13 (Cl13), looking for transcripts that showed similar expression patterns to the co-inhibitory receptor, PD-1. Interestingly, we found that the expression pattern of SIRPα closely followed that of PD-1.

SIRPα (SHPS-1, CD172a) is an inhibitory receptor whose expression was previously thought to be limited to myeloid cells, hematopoietic stem cells, and neurons. The binding of macrophage SIRPα to its widely expressed ligand, CD47, induces an inhibitory signal for phagocytosis, a “don't eat me” signal that prevents the phagocytosis of healthy cells. Mice with genetic inactivation or mutation of SIRPα have numerous abnormalities including impairment of phagocyte migration, dendritic cell homeostasis, bone cell differentiation, kidney function, and IL-17 and IFNγ production. Phagocytes from SIRPα mutant mice also have enhanced respiratory bursts. Cancer cells upregulate CD47 to evade macrophage clearance by inhibiting phagocytosis. Positive roles for SIRPα have also been described including a mechanistic role in the fusion machinery of macrophages and the binding of antigen presenting cells to bovine CD4+ T cells during priming.

Unexpectedly, we found that SIRPα expression was inducible on a subset of CD8⁺ T cells during immune activation, and that its expression was coincident with PD-1 expression, but more limited. Based on its role as a co-inhibitory receptor on macrophages and its expression on PD-1^{hi} CD8⁺ T cells, we expected that SIRPα might play an inhibitory role in exhausted T cells. Indeed, the SIRPα⁺ subset had high expression of inhibitory molecules, but this was counter-balanced by high expression of co-stimulatory molecules. Furthermore, the SIRPα⁺ subset had high levels of cytotoxic granules, displayed evidence of recent cytolytic activity (CD107a⁺), and were more cytotoxic ex vivo than the SIRPα⁻ subset. In vivo CTL experiments indicated that SIRPα interactions with CD47 were important for optimal cytolytic activity. Thus, SIRPα marks the subset of PD-1⁺ CD8⁺ T cells that retains antiviral activity during chronic FV infection.

### Results

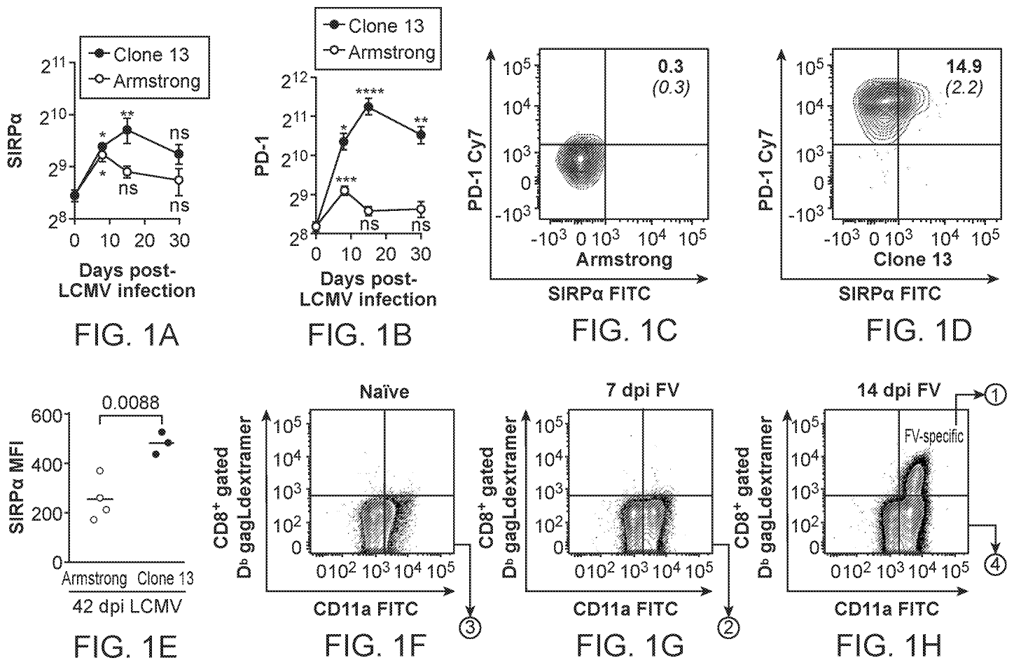

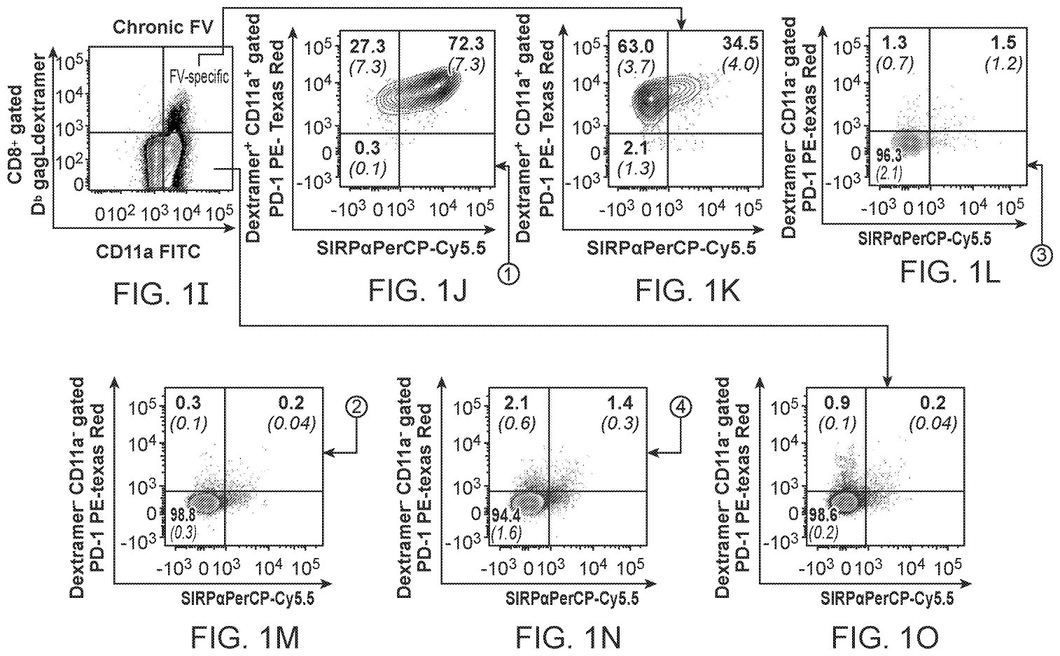

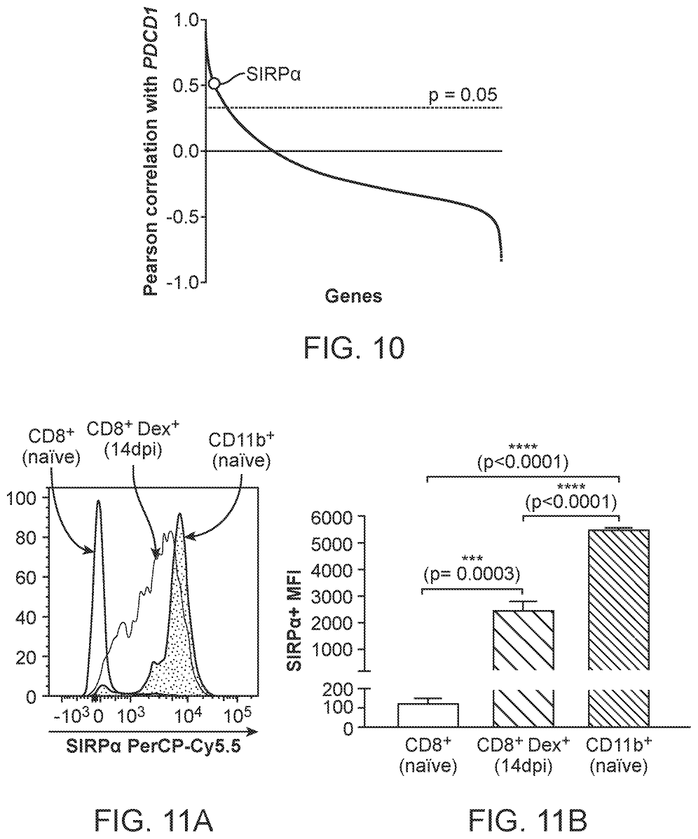

SIRPα is expressed on CD8÷ T cells during LCMV infection. To identify cell surface markers that could identify unique subsets of exhausted CD8⁺ T cells, an analysis of publicly available microarray data was performed on TCR transgenic LCMV-specific CD8⁺ T cells that had been adoptively transferred into WT mice infected with either the Armstrong (Arm) strain of LCMV (causes only acute infections) or the 0113 strain (progresses to chronic infections). We identified Sirpa as a gene of interest because it showed an expression pattern similar to PD-1 over time and had sustained upregulation during 0113 chronic infection compared to more transient expression with Arm infection (FIG. 1a, b). 20776 genes were analyzed for correlated expression with Pdcd1 and Sirpa ranked in the 97^{th} percentile during acute and chronic infection (FIG. 10). Sirpa was of special interest because it had been shown to be important in innate immunity but was not known to be expressed on CD8⁺ T cells or other adaptive immune cells. Furthermore, the sustained expression of Sirpa on CD8⁺ T cells late after infection with Cl13 suggested that it might identify an interesting subset of cells during exhaustion. Protein expression was verified by flow cytometry on LCMV-specific, transgenic CD8⁺ T cells at 42 days post-infection when CD8⁺ T cell responses to Arm would have contracted but responses to Cl13 would be largely exhausted and express PD-1. Over 90% of the transgenic CD8⁺ T cells remaining after Arm infection were PD-1 low and SIRPα⁻ (FIG. 1c). In contrast, over 95% of the transgenic CD8⁺ T cells remaining after Cl13 infection were PD-1 high and a significant subset expressed SIRPα (FIG. 1d). The mean fluorescence intensity of SIRPα expression was significantly higher on the CD8⁺ T cells responding to the chronic Cl13 strain compared to acute Arm (FIG. 1e).

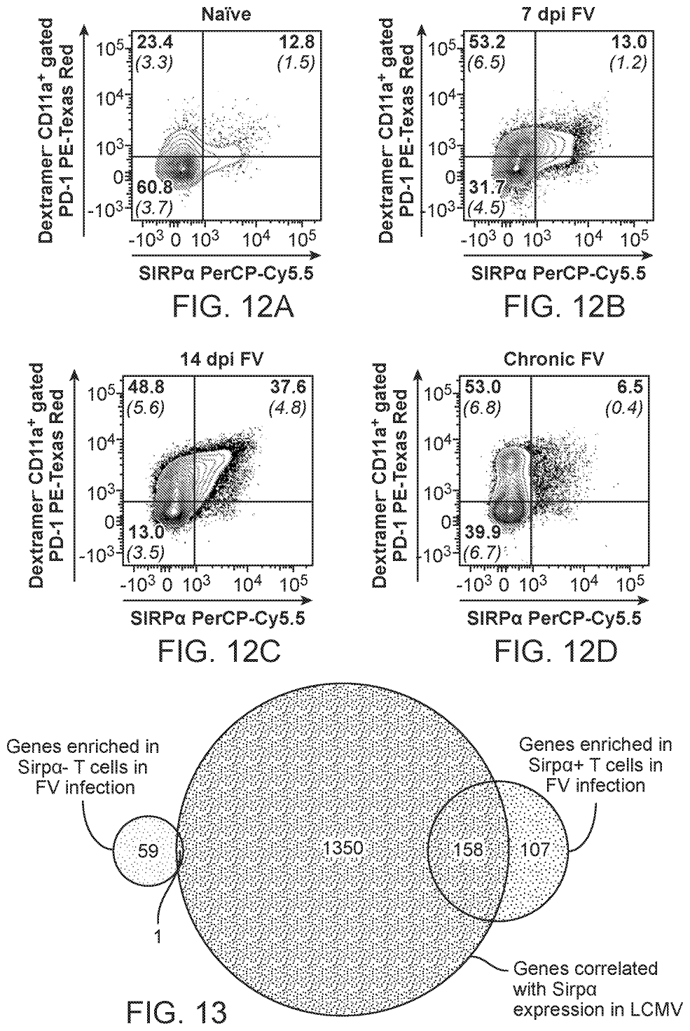

SIRPα upregulation during acute and chronic Friend virus infection. To further examine SIRPα expression on CD8⁺ T cells during chronic infection, we analyzed naïve controls (FIG. 1f) and mice infected with Friend retrovirus (FV) during early acute infection (7 dpi) (FIG. 1g), late acute infection when T cell responses peak (14 dpi) (FIG. 1h), and chronic infection (>6 wpi) (FIG. 1i) when T cells are exhausted. FV-specific CD8⁺ T cells were stained with dextramers specific for the immunodominant CD8⁺ T cell epitope, gagL, and with the activation marker CD11a (FIG. 1f-i). Subpopulations gated for these markers as indicated by quadrants with arrows were then analyzed for expression of PD-1 and SIRPα. Consistent with previous reports, almost all CD8⁺ T cells from naïve mice were SIRPα⁻ and did not stain with FV-specific dextramers (FIG. 1f, l). At 7 dpi there were still very few dextramer⁺ cells, but by 14 dpi there was a distinct subpopulation of activated, dextramer⁺ cells (FIG. 1h) that expressed PD-1 and a large majority of which (mean=72.3%) also expressed SIRPα (FIG. 1j) albeit at lower levels than macrophages (FIG. 11). During chronic infections, dextramer⁺ cells were preserved (FIG. 1i), expressed PD-1 (FIG. 1k), and about one third of them also expressed SIRPα (mean=34.5%) (FIG. 1k). Cells high in SIRPα expression were generally also high in PD-1 expression (FIG. 1j, k). SIRPα was also expressed on activated (CD11a⁺) CD8⁺ T cells responding to other FV peptides (FIG. 12). Thus, SIRPα was expressed on activated CD8⁺ T cells during both acute (FIG. 1j) and chronic FV infection (FIG. 1k and FIG. 12) while non-activated cells remained predominantly negative (FIG. 1l-o).

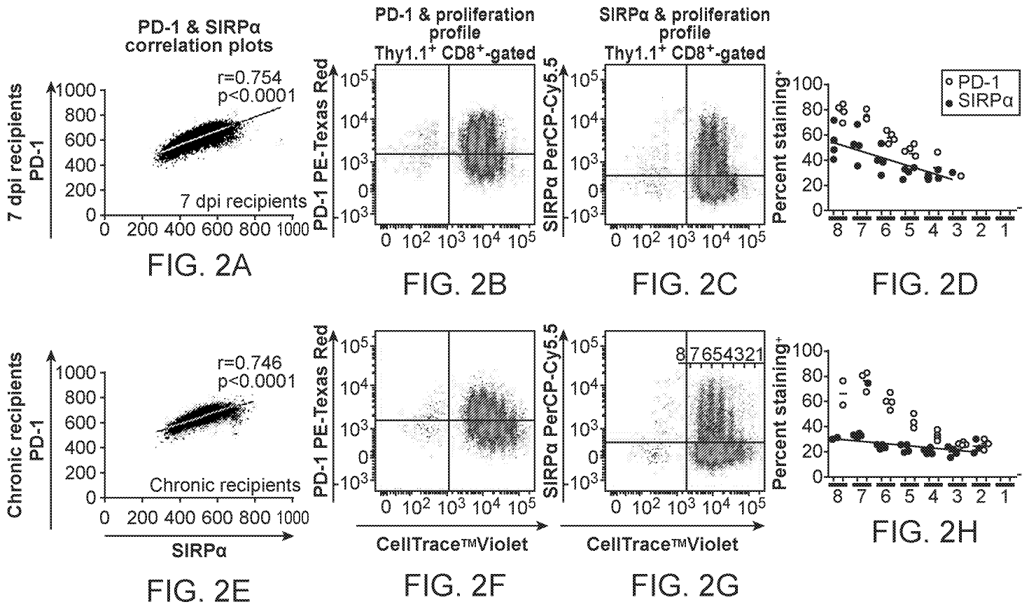

SIRPα upregulation after cell division. To examine the kinetics of SIRPα upregulation during FV infection, adoptive transfer experiments were performed using labeled, FV-specific TCR transgenic CD8⁺ T cells carrying the Thy1.1⁺ genetic marker. Naïve donor cells were adoptively transferred into Thy1.2⁺ mice that were either acutely (7 dpi) or chronically (>6 wpi) infected with FV. Such cells adoptively transferred into acutely infected mice are highly functional whereas they rapidly become dysfunctional upon transfer into chronically infected recipients. Three days after transfer the donor cells were analyzed for expression of SIRPα, PD-1 and proliferation (dilution of fluorescent signal). Pearson correlation analyses showed highly significant correlations between expression of PD-1 and SIRPα in both acutely and chronically infected mice (FIGS. 2a and 2e). Both PD-1 and SIRPα expression were induced during cell division (FIG. 2b, c, f, g) and the expression data were quantified for multiple mice at each cell division (FIG. 2d, h). In chronically infected mice SIRPα expression was rapidly induced in about 20% of the transferred cells and slightly but significantly increased to about 35% (FIG. 2h), similar to the endogenous subset (FIG. 1k). By contrast, cells transferred into acutely infected mice showed increasing levels of SIRPα expression throughout all divisions (FIG. 2d). Thus, donor cells from the same pool of naïve SIRPα⁻ cells had much different levels and kinetics of SIRPα induction dependent on whether they were transferred into acutely infected or chronically infected mice.

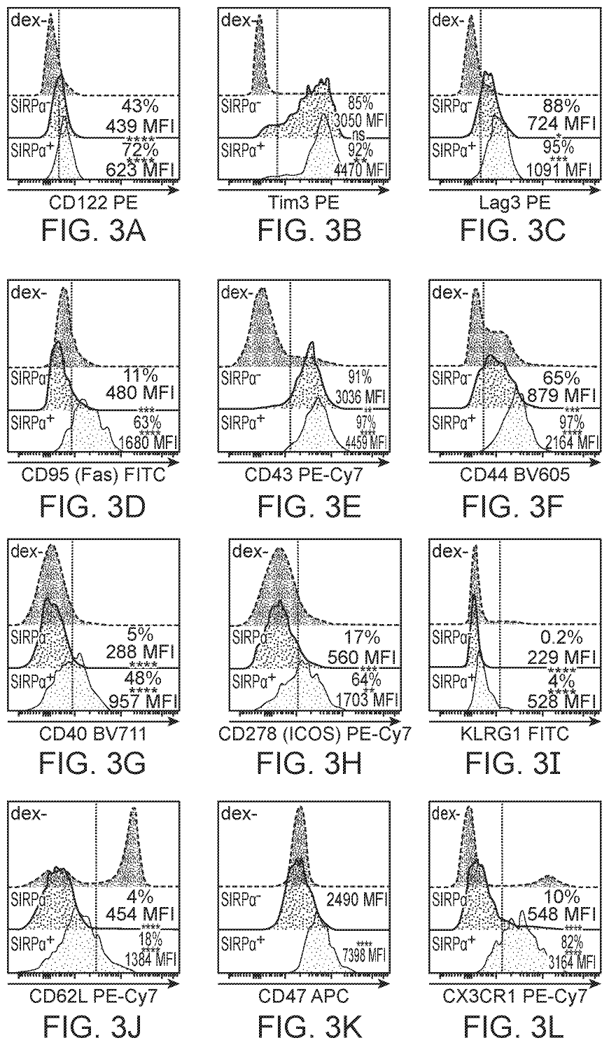

Distinct phenotype of SIRPα⁺ CD8÷ T cells. To determine if the FV-specific PD-1/SIRPα double positive CD8⁺ T cells from chronically infected mice comprised a subset of cells with a distinct phenotype, the expression of additional markers was examined by flow cytometry. The PD-1⁺ SIRPα⁺ CD8⁺ T cells from chronically infected mice also expressed high levels of the co-inhibitory receptors Tim3 and Lag3, CD95 (Fas), which leads to apoptosis upon ligand binding, and IL-2Rβ chain (CD122), which helps drive a PD-1^{hi} phenotype (FIG. 3a-d). This expression pattern would suggest an exhausted phenotype except that these cells also expressed high levels of the activation/co-stimulatory molecules, CD43, CD44, CD40 and CD278 (ICOS) (FIG. 3e-h). Neither the SIRPα⁻ or the SIRPα⁺ subsets showed high expression of the terminal differentiation marker, KLRG1, but mean expression was twice as high on the SIRPα+ subset as the SIRPα⁻ subset (FIG. 3i). The expression of CD62L (L-selectin lymphoid homing receptor) on SIRPα⁺ CD8⁺ T cells, which is downregulated during activation but returns on memory T cells was intermediate between SIRPα⁻ CD8+ T cells and naïve CD8⁺ T cells (FIG. 3j). SIRPα⁺ CD8⁺ T cells expressed high levels of CD47, the ligand for SIRPα (FIG. 3k), which is interferon-inducible gene and its increased expression marks functional, long-lived memory CD4⁺ T cells. CX₃CR1, reported to identify granzyme B positive, cytotoxic memory CD8⁺ T cells was also much higher on SIRPα⁺ than SIRPα⁻ CD8⁺ T cells (FIG. 3l). Thus, the PD-1⁺ SIRPα⁺ CD8⁺ T cells had a unique surface marker expression phenotype with high expression of both co-inhibitory and co-stimulatory molecules, and characteristics of a functional memory phenotype.

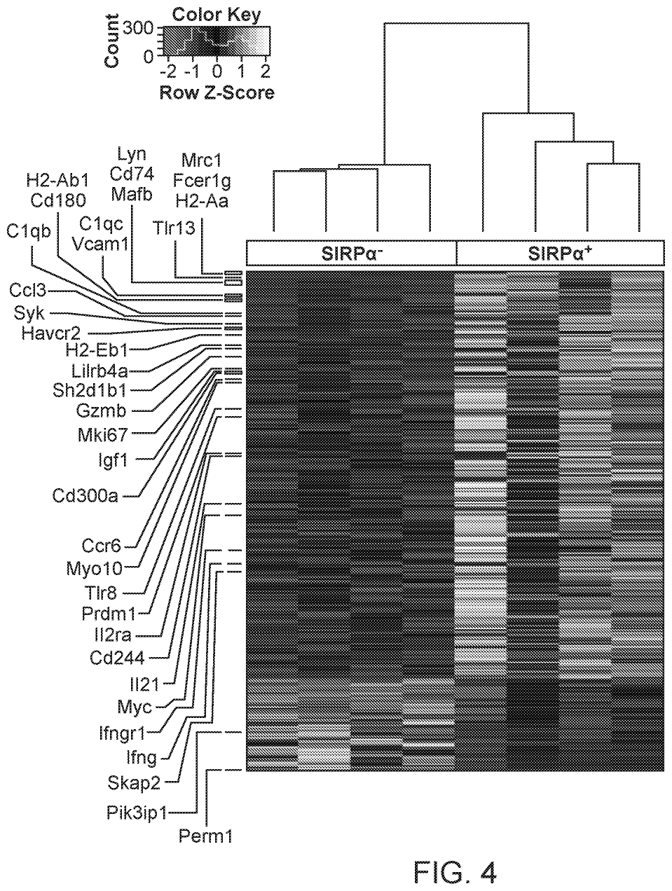

SIRPα⁺ CD8⁺ T cells have a unique transcriptional profile. To gain a broad perspective of the differences between CD8⁺ T cells expressing SIRPα or not, whole transcriptome shotgun sequencing (RNA-SEQ) was performed on cell-sorted populations of splenic SIRPα⁻ and SIRPα⁺ FV-specific TCR transgenic CD8⁺ T cells that had been adoptively transferred into FV chronically infected mice two weeks earlier. A total of 325 transcripts were differentially expressed at a significant level between the subpopulations, and 82% of the differentially expressed transcripts were upregulated in the SIRPα⁺ population. Granzyme B (Gzmb), Ki-67 (Mki67) IFNγ (IFNG) and the inflammatory chemokines CCL3 and CCL4 were significantly upregulated in the SIRPα⁺ subset (FIG. 4), which is consistent with these cells expressing markers of activation and co-stimulation (FIG. 3). Analysis of the top 100 most differentially upregulated genes by gene set enrichment analysis (ToppFun) revealed that the top biological process upregulated by SIRPα⁺ CD8⁺ T cells was positive regulation of the immune system followed by active proliferation (Table 1).

| | | | | FDR | FDR | | Genes | Genes in |
|---|---|---|---|---|---|---|---|---|
| | ID | Name | pValue | B&H | B&Y | Bonferroni | from Input | Annotation |
| 1 | GO:0002684 | positive regulation of | 9.134E−12 | 2.032E−8 | 1.748E−7 | 2.787E−8 | 25 | 976 |
| | | immune system process | | | | | | |
| 2 | GO:0051301 | cell division | 1.332E−11 | 2.032E−8 | 1.748E−7 | 4.064E−8 | 21 | 668 |
| 3 | GO:0002683 | negative regulation of | 3.146E−11 | 3.199E−8 | 2.752E−7 | 9.598E−8 | 17 | 420 |
| | | immune system process | | | | | | |
| 4 | GO:0022402 | cell cycle process | 1.033E−10 | 7.709E−8 | 6.630E−7 | 3.153E−7 | 28 | 1385 |
| 5 | GO:0000278 | mitotic cell cycle | 1.386E−10 | 7.709E−8 | 6.630E−7 | 4.228E−7 | 24 | 1015 |

Intriguingly, the next highest biological process was negative immune regulation. Thus, the SIRPα⁺ T cells transcribed numerous genes capable of both immune activation and inhibitory functions with a skewing towards activation. Comparison of the genes that correlated with SIRPα expression in LCMV-specific CD8⁺ T cells with the genes significantly upregulated in FV-specific PD1⁺SIRPα⁺ CD8 T cells identified 158 genes that were shared (FIG. 13). The most downregulated gene in the SIRPα⁺ subset was PERM1, an inducer of mitochondrial biogenesis and oxidative phosphorylation, typically utilized by exhausted T cells whereas effector T cells downregulate mitochondrial biogenesis in favor of the glycolytic pathway. Another highly downregulated gene was an inhibitor of T cell activation, Pik3ip1. Thus, the phenotyping (FIG. 3) and transcriptional profiling (FIG. 4) results indicated that SIRPα identified a unique subset within the exhausted population of CD8⁺ T cells with preserved effector function.

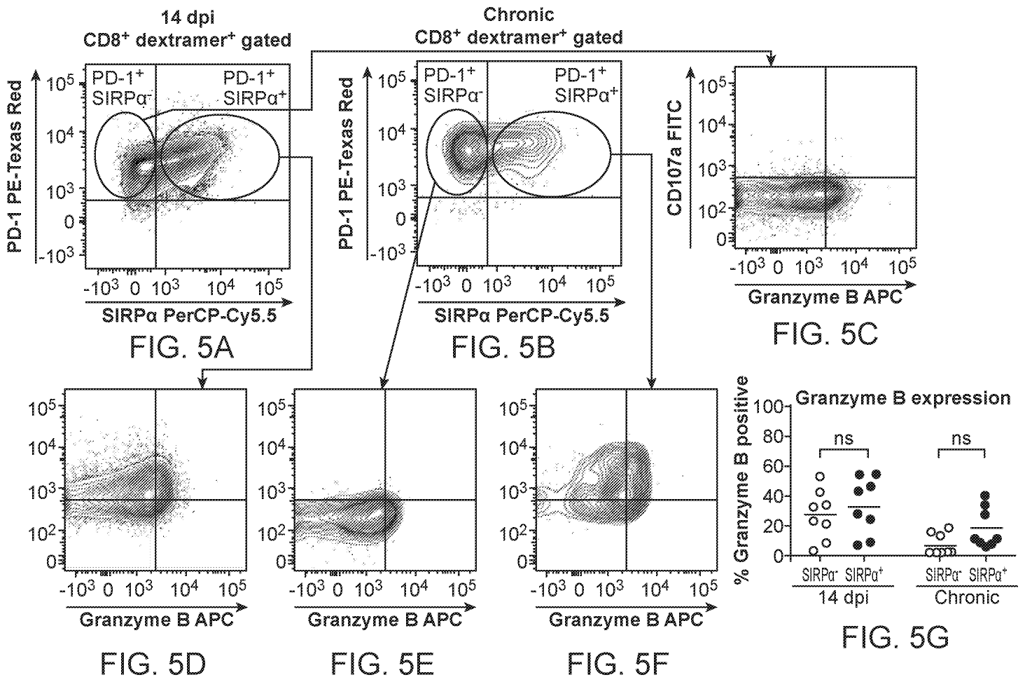

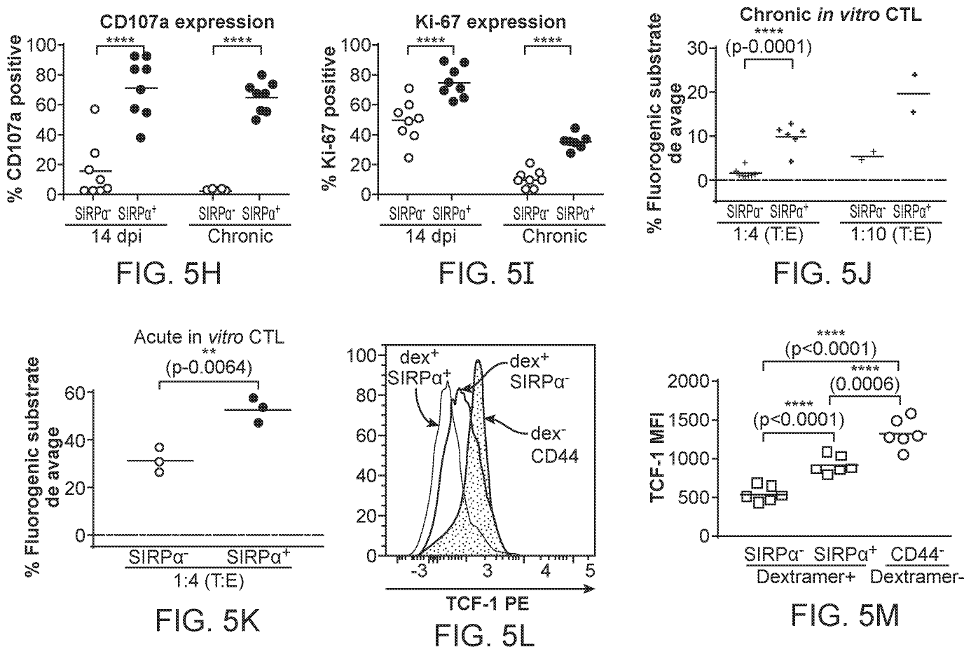

SIRPα expression associated with in vivo effector function. To determine whether the function of the SIRPα⁺ subset differed from the SIRPα⁻ subset, FV-specific (dextramer⁺) CD8⁺ T cells from acutely (FIG. 5a) and chronically (FIG. 5b) infected mice were stained for intracellular expression of granzyme B and surface expression of CD107a, an indicator of recent cytolytic activity. Both the SIRPa⁺ and SIRPα⁻ subsets had cells expressing granzyme B (FIG. 5c-g). Importantly, almost no SIRPα⁻ cells expressed CD107a while more than half of the SIRPα⁺ cells were CD107a⁺, indicating that they had recently undergone exocytosis (FIG. 5c-f, h). Similar results were found from both acutely and chronically infected mice. In acutely infected mice, a large percentage of both the SIRPα⁺ and SIRPα⁻ subsets had recently proliferated (Ki-67⁺), although the proportion in the SIRPα⁺ subset was significantly higher (FIG. 5i). In chronically infected mice, very few SIRPα⁻ cells were Ki-67⁺ whereas a mean of approximately 35% of the SIRPα⁺ subset was Ki-67⁺ (FIG. 5i). Thus, the SIRPα⁺ subset appeared more functional in both cytolytic activity and proliferative capacity than the SIRPα⁻ subset, confirming the transcriptional profile results provided by the RNA-SEQ analysis and ToppFun analysis (FIG. 4, Table 1).

In vitro CTL killing by SIRPα⁺ CD8⁺ T cells. A direct test of cytolytic activity was done using an in vitro killing assay to compare the SIRPα⁻ and SIRPα⁺ subsets. To obtain sufficient cells for the assay and to avoid stimulating sorted cells by crosslinking with dextramers, we performed adoptive transfers of genetically labeled (Thy1.1⁺), FV-specific, TCR transgenic CD8⁺ T cells specific for the immunodominant FV gag peptide. The cells were adoptively transferred into chronically infected recipients where they were allowed to proliferate and become exhausted for 13-15 days. They were then harvested and FACS-sorted into SIRPα⁻ and SIRPα⁺ subpopulations and co-cultured with either FV-gag peptide-loaded target cells or control cells. As expected for CD8⁺ T cells from a chronic infection, the in vitro CTL activity was low but significantly more FV-specific killing was observed with the SIRPα⁺ CD8⁺ T cells than with the SIRPα⁻ subset (FIG. 5j). For comparison, SIRPα⁺ and SIRPα⁻ CD8⁺ T cell effectors taken from acutely infected mice displayed much higher killing frequencies than cells from chronic infections (FIG. 5k), but consistent with the chronic infection results, more killing was observed with the SIRPα⁺ subset compared to the SIRPα⁻ subset. Thus, during both acute and chronic FV infections, expression of SIRPα⁻ correlated with enhanced cytolytic ability (FIG. 5j, k) and proliferative capacity (FIGS. 4 and 5i), suggesting that SIRPα identified cells that sustained an antiviral response during chronic infection. Such a role was recently associated with the transcription factor, T cell factor-1 (TCF-1) and we observed significantly higher intracellular TCF-1 expression in the SIRPα⁺ CD8⁺ T subset than the SIRPα⁻ subset (FIG. 5l, m).

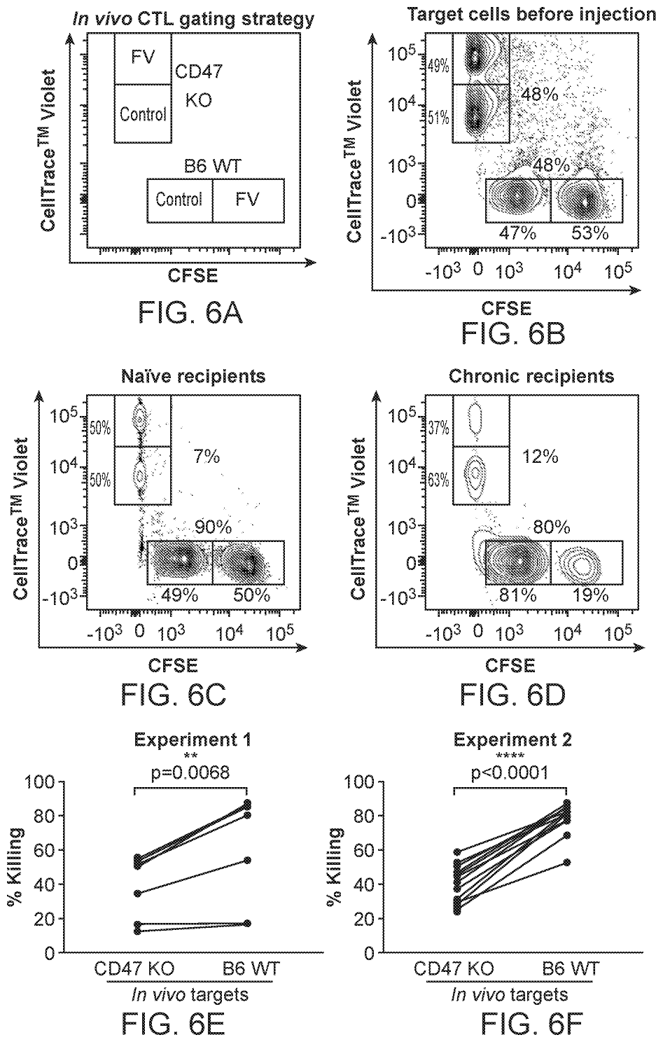

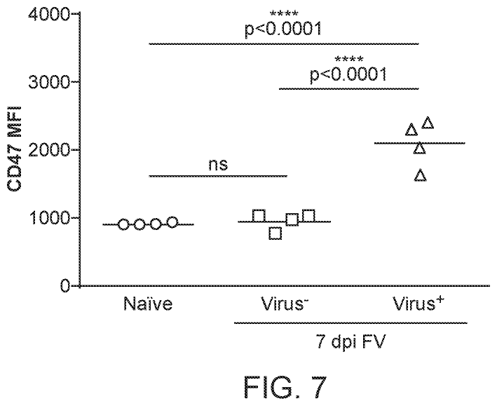

CD47⁺ targets are more efficiently killed in vivo. To confirm that cytolytically active CTLs were present in chronically infected mice and to ascertain whether SIRPα was playing a functional role in that activity, an in vivo CTL killing experiment was performed using viral peptide-loaded target cells that either expressed CD47, the ligand for SIRPα, or had a gene inactivation of CD47. Target cells from both WT and CD47 null genotypes, either FV peptide-loaded or control-treated, were differentially labeled with fluorescent stains (FIG. 6a), and all four types of target cells were adoptively transferred at equivalent numbers (FIG. 6b) into naïve or chronically infected mice (FIG. 6c, d). Spleens were harvested 6 hours after transfer and analyzed by flow cytometry for killing. CD47 null target cells were susceptible to macrophage-mediated phagocytosis regardless of loading with cognate peptide but no virus-specific killing of targets was observed in naïve mice as both control targets and peptide-loaded targets remained at the starting ratio of 50:50 (FIG. 6c). In contrast, virus-specific killing was observed in chronically infected mice (FIG. 6d), which was quantified in two separate experiments. In the first experiment, four of the six chronically infected mice tested displayed CD8⁺ CTL activity, and the virus-specific killing was significantly greater in wild type targets than in CD47 null targets (FIG. 6e). In the second experiment, all fourteen mice displayed CTL activity, which was again significantly greater against the wild type targets compared to the CD47 null targets (FIG. 6f). Interestingly, compared to uninfected cells from an FV-infected mouse, the infected cells significantly upregulated expression of CD47 (FIG. 7). Thus, SIRPα-CD47 ligation was not required for cytolysis in vivo, but it significantly enhanced cytolysis.

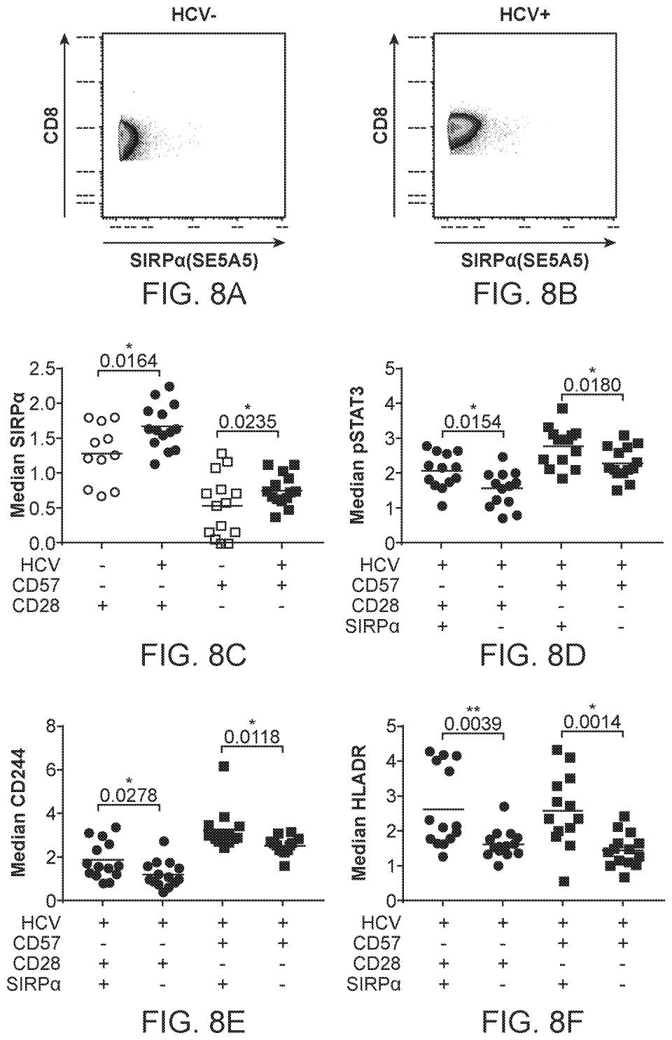

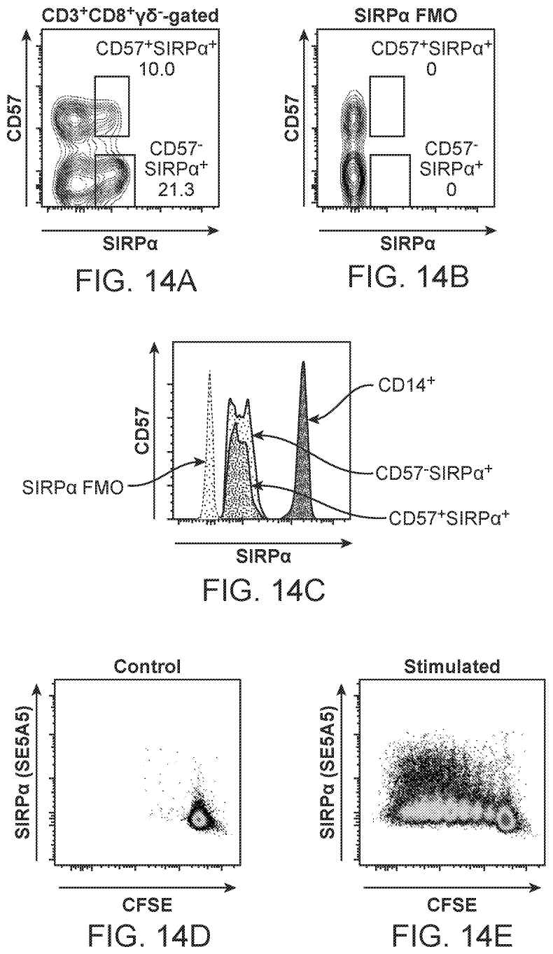

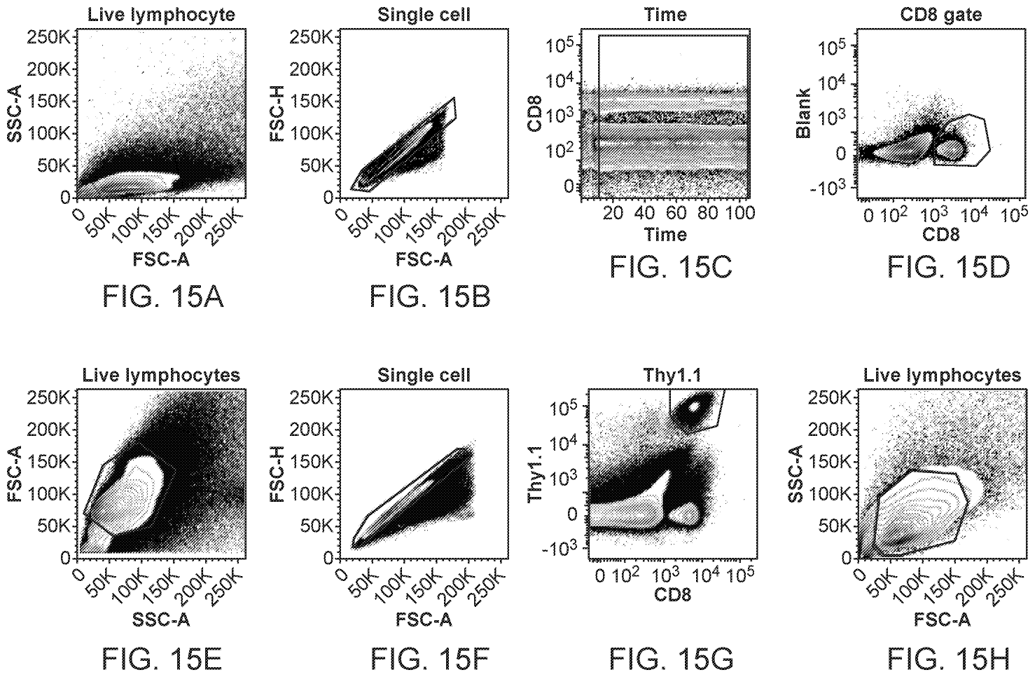

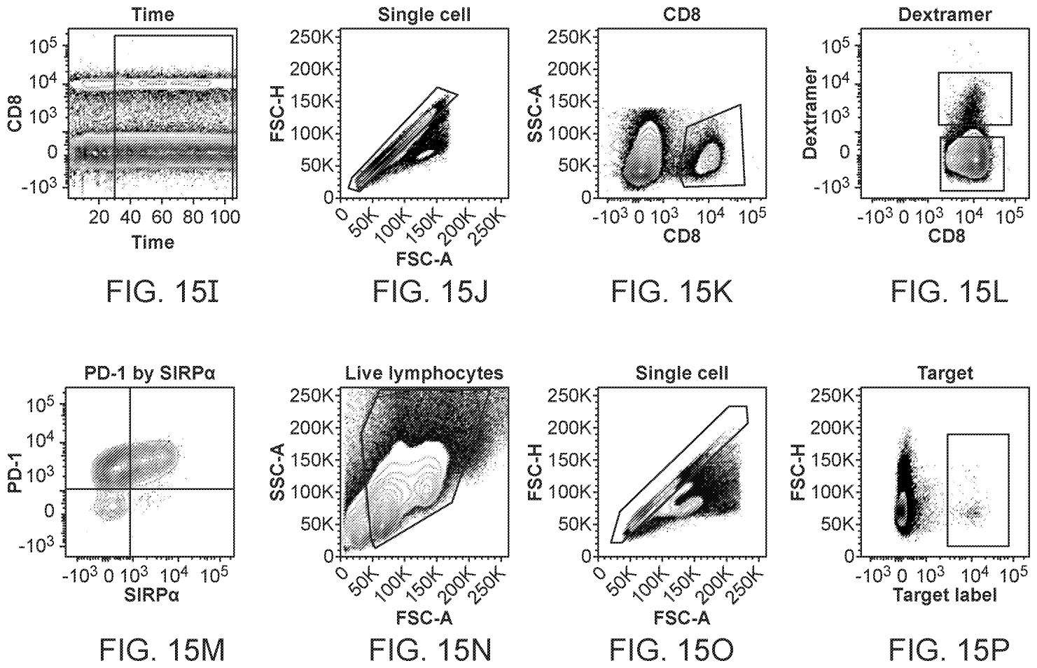

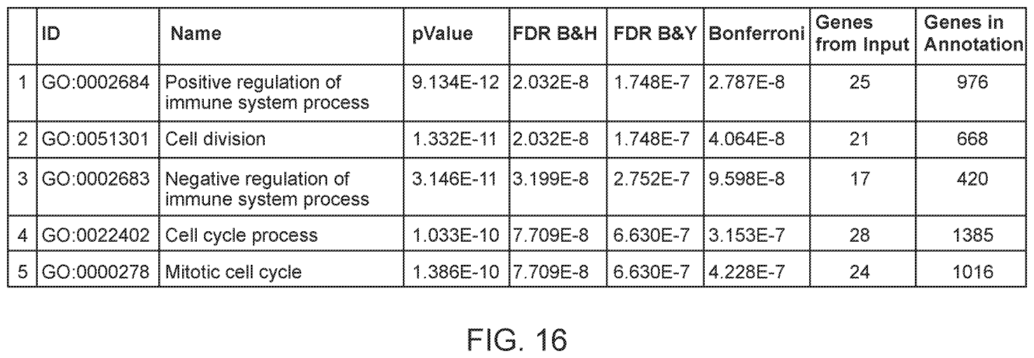

CD8⁺ T cells from human HCV patients upregulate SIRPα. To determine whether SIRPα expression could also be found on human T cells during a chronic viral infection, CD8⁺ T cells from healthy controls or patients with chronic Hepatitis C virus (HCV) were examined using CyTOF, flow cytometry that uses heavy metal ion-tagged antibodies. In CD8⁺ T cells from both HCV uninfected and infected patients, the main subset was SIRPα negative (FIG. 8a,b). However, in HCV patients there was a subpopulation of CD8⁺ T cells with increased expression of SIRPα (FIG. 8a,b and FIG. 14a,b). We analyzed CD57 and CD28 markers because chronic antigenic stimulation of human CD8⁺ T cells is associated with the down-regulation of costimulatory CD28 and upregulation of CD57. The CD57⁺ CD28⁻ subset is increased in HCV patients. Although this subset is heterogenous, it is generally associated with a reduced state of function and proliferation. SIRPα expression was significantly higher on CD8⁺ T cells from HCV-infected individuals compared to controls in both the functional CD57⁻ CD28⁺ subset as well as the CD57⁺ CD28⁻ subset (FIG. 8c). Samples from one patient were also tested by flow cytometry and an example of the data and comparison with SIRPα expression on macrophages is shown (FIG. 14c). Thus, SIRPα is also expressed on human CD8⁺ T cells and is upregulated during chronic HCV infections. SIRPα⁺ cells from both CD57⁻ and CD57⁺ subsets also had higher levels of phosphorylated STAT3, CD244/264 and HLA-DR indicating a higher activation status compared to their SIRPα⁻ counterparts (FIGS. 8d, e and f). These results are consistent with SIRPα⁺ marking a subset of functional CD8⁺ T cells. Furthermore, stimulation human PBMCs for 5 days led to significant upregulation of SIRPα on proliferating CD8⁺ T cells in comparison to unstimulated controls (FIG. 14d, e).

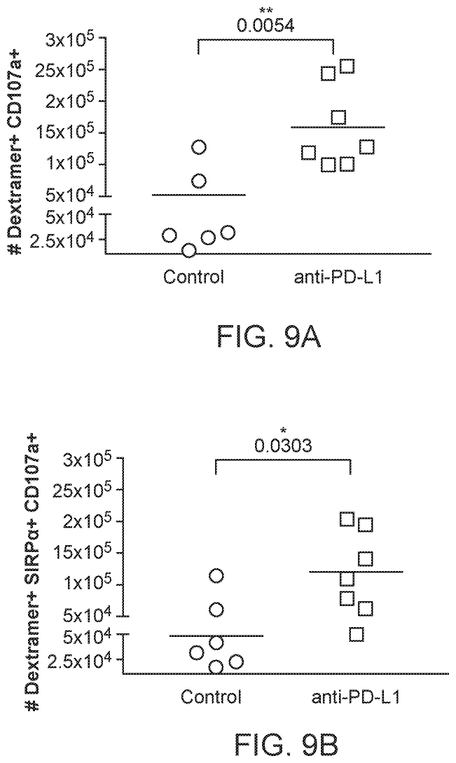

PDL-1 blockade expands SIRPα⁺ CD8⁺ T cells. Of interest in treating chronic infections and cancer are immune checkpoint inhibitors, such as anti-PD-1 or anti-PD-L1, which can reinvigorate exhausted T cell responses. We treated FV-chronically infected mice with anti-PD-L1 and observed a significant expansion of FV-specific (Dextramer⁺) recently cytolytic (CD107a⁺) CD8⁺ T cells (FIG. 9a). An average of 80% of the recently cytolytic CD8⁺ T cells were also SIRPα⁺ (FIG. 9b) indicating that either the SIRPα⁺ subset specifically expanded or that the expanded subset of cytolytic cells upregulated SIRPα. Thus, expression of SIRPα can be used to determine whether immune checkpoint inhibitor therapy successfully expanded functional CD8⁺ T cells.

Until now, SIRPα has been considered to primarily be an inhibitory signaling receptor expressed predominantly on myeloid cells in the hematopoietic compartment. The results presented here present a more complex picture. We confirm that SIRPα has little or no expression on naïve T cells, as previously shown in mice, rats and humans. However, we now show that SIRPα is expressed on activated CTL during acute viral infection. Such expression may have previously been missed due to examination of only naïve cells. In addition to the expanded cell-specific expression profile, it is possible that SIRPα signaling in CD8⁺ T cells may not be negative. During both acute and chronic FV infections, almost all CD8⁺ T cells that showed evidence of recent cytolytic activity (CD107a⁺) were also SIRPα⁺ (FIG. 5h). Compared to the SIRPα⁻ subset, the SIRPα⁺ subset was also significantly more proliferative (Ki-67⁺) (FIG. 5i), expressed higher levels of TCF-1 (FIG. 5m), had higher expression of IFNγ message (FIG. 4), and transcribed significantly more genes indicative of immune activation (FIG. 4). Furthermore, cell-sorted SIRPα⁺ CD8⁺ T cells from chronically infected mice had greater in vitro cytotoxicity than the SIRPα⁻ cells from the same mice (FIG. 5j). However, SIRPα might simply mark the active CTL subset rather than positively regulating CD8+ T cell functions. CTL targets lacking CD47 were nevertheless killed by CTL in vivo, albeit to a significantly lower level than targets expressing CD47 (FIG. 6e, f). These results indicate that SIRPα-CD47 interactions are involved in the cytolytic process, but do not address whether SIRPα acts as a positive or negative regulator of functional CTL development.

What is most evident and novel from the data is that cell-specific SIRPα expression is not as limited as previously thought, that it is expressed on the most proliferative and functionally active subset of CD8⁺ T cells in both acute and chronic infections, and that it is involved in the cytolytic process. As such, SIRPα allow practitioners to follow the expansion or contraction of the functional subset during immunotherapy with relevance not only to infections, but also cancer and autoimmune diseases. We find that SIRPα protein surface expression is increased not only in activated mouse T cells, but also in activated human T cells, suggesting that this may be a conserved marker of active CD8⁺ T cells. The elevated SIRPα levels on CD8⁺ T cells from patients with chronic HCV infection was most pronounced on the CD57-CD28⁺ subset.

It is worth considering how SIRPα-CD47 interactions might be involved in the cytolytic process in vivo since cognate target cells that did not express CD47 were killed less effectively than wild type target cells in multiple in vivo CTL assays (FIG. 6e, f). SIRPα is capable of delivering activating as well as inhibitory signals depending on the context including the presence of adapter proteins such as Skap2 and GRB2, and phosphatases such as SHP1 and SHP2. No evidence of differential expression of SHP1 or SHP2 was found in the RNA-SEQ analysis, but Skap2 transcription was increased 2.8 fold in the SIRPα⁺ subset. Interestingly, one of the most highly overexpressed genes in the SIRPα⁺ subset (20× increase) was Lyn. Lyn is a tyrosine protein kinase with a role in regulating cell activation, and like SIRPα, it has an inhibitory role in myeloid cells. In B cells, Lyn phosphorylation initiates an activation cascade indicating that like SIRPα, it can deliver either inhibitory or activating signals in a context-dependent manner. Alternatively, it is possible that SIRPα-CD47 interactions simply stabilize cell to cell contacts and the cytolytic synapse. The spanning distance of end-to-end bound CD47-SIRPα complexes (˜14 nm, similar to TCR-MHC, CD28-CD86 and CD40-CD40L) suggests that significant binding between a target and effector cell would take place predominantly in immunological synapses where abundant bulkier cell surface proteins such as CD43 and CD45 that can sterically hinder more short range interactions, are redistributed outside of the cytolytic synapse. The strength of the interactions between cells is influenced not only by the affinity between the receptors and ligands, but also by their density. Thus, the high expression of SIRPα on CTL combined with up-regulated CD47 on infected targets (FIG. 7) could have a significant impact on the strength and duration of interactions within the immunological synapse as has been previously suggested.

Prior to these experiments it was known that virus-specific CD8⁺ T cells were sustained in FV-chronically infected mice, albeit at low numbers (˜1-3% of CD8⁺ T cells). It is now apparent that the FV-specific CD8⁺ T cells in mice with chronic FV are heterogeneous with respect to function, and that there is a cytolytically active subset that can be identified by cell surface expression of SIRPα. As discussed, this active subset displays high expression of both co-stimulatory and co-inhibitory molecules (FIG. 3a-h). Immune checkpoint inhibition by anti-PD-L1 produced an expanded population of CTL, the large majority of which expressed SIRPα (FIG. 9). In chronic LCMV infections the degree of expression of multiple co-inhibitory receptors has been shown to correlate with the severity of T cell dysfunction, but expression of co-inhibitory receptors is not specific to exhausted cells and occurs during T cell activation as well. Thus, it is not possible to differentiate dysfunctional T cells from activated T cells based only on the expression of co-inhibitory receptors. Recently there has been shown to be a great deal of heterogeneity in the level of dysfunction of CD8⁺ T cells in an exhausted setting such as within a tumor or in a chronic infection. A detectable level of function and virus control persists in chronic viral settings as evidenced by the fact that CD8⁺ T cell escape variants arise in chronic HIV infections and that viral titers increase following depletion of CD8⁺ T cells in SIV-infected macaques. Depletion of CD8⁺ T cells in mice with chronic FV infections does not produce virus relapse, but this is likely due to compensatory mechanisms by antiviral CD4⁺ T cells and does not indicate that the residual CD8⁺ T cells exert no control over chronic infection. The SIRPα positive and negative CD8⁺ T cell subsets during exhaustion did not have significantly different expression levels of Tbet, EOMES, CTLA4, or Bcl2 (Supplemental dataset 1). Although the SIRPα⁺ subset displayed higher levels of CD44, Ki67, TCF-1 and CD62L suggesting a functional memory phenotype, they also displayed high levels of co-inhibitory receptors indicating a different phenotype than previously described for exhausted or functional cells.

We have shown that the expression of SIRPα on CD8⁺ T cells is diagnostic for the presence of active CTLs, even in exhausted settings. Thus, SIRPα⁺ CD8⁺ T cells are interesting targets for immunotherapy, and can be specifically expanded or further activated to kill chronically infected cells and/or tumors. The current results suggest that while activating macrophage antitumor phagocytosis, CD47 blockade might also inhibit CD8⁺ T cell antitumor activity. However, such inhibition might be overcome using PD-1 blockade, a cancer immunotherapy currently thought to function primarily via CD8⁺ T cell activation. Interestingly, it was recently shown that, similar to tumor-infiltrating T cells, tumor-associated macrophages also express PD-1 co-inhibitory receptors. Thus, cancer therapies targeting PD-1 or its ligand could be activating macrophage functions as well as T cells. A combination of PD-1 and CD47 blockade could have synergistic effects by potentiating the antitumor activity by both macrophages and T cells.

### Materials and Methods

Mice, viruses, infection, and tissue harvest. For LCMV studies, female 4-6 week old C57BL/6J mice from NCI and Thy-1.1⁺ P14 TCR transgenic mice that recognize the H-2Db gp33 epitope were used where indicated. Mice were intraperitoneally (i.p.) infected with 2×10⁵ plaque forming units (p.f.u.) LCMV Armstrong (Arm)—which causes an acute infection—or intravenously (i.v.) with 2×10⁶ p.f.u. LCMV Clone 13 (Cl13)—which causes a chronic infection. The use of all animals was conducted in accordance with Yale University IACUC guidelines. For Friend virus studies, mice were female (C57BL/10×A.BY) F1 (Y10) (H-2^{b/b}, Fv1^{b}, Rfv3^{r/s}, Fv2^{r/s}) and FV-specific Thy1.1⁺ CD8.TCR transgenic mice between 12-24 weeks of age at the beginning of the experiments and were bred at the Rocky Mountain Laboratories. The FV stock has been passaged in mice for more than three decades and contains three separate viruses: 1) B-tropic Friend murine leukemia helper virus (F-MuLV), which is a replication competent retrovirus; 2) polycythemia-inducing spleen focus-forming virus (SFFV), which is a defective retrovirus that is packaged by F-MuLV-encoded virus particles; and 3) lactate dehydrogenase-elevating virus, an endemic murine nidovirus related to coronaviruses. Mice were infected by i.v. injection of 0.2 mL PBS containing 1500 spleen focus-forming units of FV complex. Mice were considered chronically infected at 6 weeks post-infection when F-MuLV levels stabilize at approximately 10⁴ infectious centers per spleen. Splenocytes were isolated by tissue homogenization through a 100-μm filter and RBCs were removed using lysis buffer (0.15 M NH₄Cl, 10 mM KHCO₃, 0.1 M EDTA). Mice were treated in accordance with RML IACUC-approved animal use protocols following the regulations and guidelines of the Animal Care and Use Committee of the Rocky Mountain Laboratories and the National Institute of Health Office of Laboratory Animal Welfare.

LCMV Affymetrix. Affymetrix arrays from GSE41867 were obtained as CEL files, MAS5 normalized using the “affy” package in Bioconductor, mapped to NCB Entrez gene identifiers using a custom chip definition file and converted to MGI gene symbols. Gene expression values were mean-and-log 2-normalized prior to analysis.

Flow cytometry. For flow cytometric analysis, live lymphocytes were gated using a SSC-A and FSC-A gate. Cells were then gated by time to exclude artifact caused by erratic sample flow and by FSC-H and FSC-A to exclude doublets. The antibodies used for cell staining were purchased from BD Pharmingen, BioLegend or eBioscience, except where otherwise noted. The antibodies used for surface staining were A700- or PacBlue-anti-CD8 (53-6.7); FITC-anti-CD11a (2D7); PE-CF594-anti-PD-1(J43); PE-Cy7-anti-Thy1.1 (H1551); FITC-CD107a (1 D4B); PE-anti-Tim3 (8B.2C12); PE-anti-Lag3 (C9B7W); FITC-anti-Fas (Jo2); PE-Cy7-anti-CD43 (1B11); BV605-anti-CD44 (IM7); BV711-anti-CD40 (3/23); PE-Cy7-anti-CD278 (C398.4A); PE-Cy7-anti-CD62L (MEL-14); PE-Cy7-anti-CX3CR1 (SA011F11); APC-anti-CD47 (miap301); PerCP-Cy5.5-anti-SIRPα (P84). mAb P84 specificity is based on the following: Signal regulatory proteins (SIRPα and SIRPβ in the mouse) are expressed on neurons, hematopoietic stem cells and myeloid cells including macrophages, monocytes, granulocytes and dendritic cells. Dendritic cells, macrophages and mononcytes from mice with targeted SIRPα gene disruptions completely lose reactivity with mAb p84 (anti-SIRPα) even though their SIRPβ expression is normal. These results indicate specificity of p84 for SIRPα without cross reactivity for SIRPβ. For FV-specific H-2D^{b}/Abu-Abu-L-Abu-LTVFL staining, APC- or PE-D^{b} gagL-MHC Dextramer (Immudex, Copenhagen, Denmark) was used. For intracellular staining, cells were surfaced stained and fixed prior to permeabilization. The antibodies used for intracellular staining were PE-anti-EOMES (Dan11mag); PE-Cy7-anti-Tbet (eBio4B10); A700-anti-Ki67 (B56); PE-anti-TCF-1 (S33-966) and APC-anti-human granzyme B (GRB05; Molecular Probes). To stain for intracellular granzyme B, cells were fixed overnight in 0.5% paraformaldehyde and then permeabilized with 0.1% saponin/PBS containing 0.1% sodium azide, 0.5% BSA and 50 mM glucose. To stain for all other intracellular markers, intracellular staining was performed using the eBioscience Foxp3 kit, following the company's recommendation. To detect FV-infected cells, cells were stained with tissue culture supernatant containing monoclonal antibody (MAb 34), which is specific for F-MuLV glycosylated Gag protein. MAb 34 binding was detected with FITC-labeled goat anti-mouse IgG2b. The muliparameter data were collected with an LSRII (BD Biosciences) and analyzed using FlowJo software (version 10.2; TreeStar, Inc.).

T cell adoptive transfers for proliferation and the in vitro CTL assay. Alpha beta CD8⁺ T cells from the spleens of naïve FV-specific Thy1.1⁺ CD8.TCR transgenic mice were first isolated by magnetic bead separation (Miltenyi MACS system) following manufacturer's recommendations. For proliferation assays, the cells were then CellTrace™ violet-labeled as directed (Invitrogen). A total of 1×10⁶ CD8⁺ T cells were transferred i.v. into either acutely infected (7 dpi) or chronically FV infected recipients. At 72 hours post-transfer, the splenocytes were surface stained and then analyzed for CellTrace™ dilution. For the in vitro CTL assay, CD8⁺ T cells from naïve FV-specific Thy1.1+CD8.TCR mice were transferred i.v. into Y10 mice chronically or acutely infected with FV. After 13-15 days, CD8+ cells were purified from the spleens of these recipients using anti-CD8 paramagnetic beads and the Miltenyi MACS system following the manufacturers recommendations. Cells were then stained with PE-Cy7-anti-Thy1.1; A700-anti-CD8; PE-CF594-anti-PD-1; PerCPCy5.5-anti-SIRPα and sorted into CD8⁺ Thy1.1⁺PD-1⁺SIRPα⁻ and CD8⁺Thy1.1⁺PD-1⁺ SIRPα⁺ populations using a BD FACSAriallu. Sorted populations were ≥95% pure in all assays as determined by flow cytometry. For a negative control effectors, bead purified CD8⁺ T cells from naïve Y10 mice were used. As target cells for these assays, CD8-depleted splenocytes that were either 1% DMSO-treated (control targets) or peptide pulsed with 25 μM DbGagL peptide in 1% DMSO for 1 hr at 37° C. were used. These target cell and effector cell populations were then placed in a 2 hr in vitro cytotoxic killing assay at a 1:4 (10,000:40,000 cells) or 1:10 (10,000:100,000) target:effector ratio (T:E) for 2 hours in the substrate following recommendations from the PanToxiLux kit (Oncolmmunin, Inc.). The samples were then immediately analyzed by flow cytometry for substrate fluorescence. For data quantification, the “background” level of substrate flouresence from the DMSO control targets was subtracted from the CTL-killing level of the gag-peptide loaded targets for each individual sample.

In vivo killing assay. The target cells for these assays were CD8-depleted splenocytes from C57/BL6 RRID: IMSR_JAX:000664 wildtype mice or CD47−/− RRID: IMSR_JAX:003173 mice on the C57/BL6 background that were either 1% DMSO-treated (control targets) or peptide pulsed with 25 μM D^{b}GagL peptide in 1% DMSO for 1 hr at 37° C. All target cells were then labeled with 375 ng per mL Deep Red (Invitrogen) and then differentially labeled with either 4 or 40 μM CFSE (Invitrogen) or either 2.5 or 25 μM CellTrace™ violet (Invitrogen). Naïve control or chronically infected recipients were given an i.v. adoptive transfer of 5×10⁶ cells of each subset at approximately 25:25:25:25 ratio, as confirmed by flow cytometry of the Time Zero sample. Spleens from recipient mice were analyzed by flow cytometry after 6 hours. The percentage of killing of each population of FV pulsed cells was calculated as follows: 100−([% peptide pulsed in infected divided by % unpulsed in infected) divided by (% peptide pulsed in uninfected divided by % unpulsed in uninfected)]×100).

In vivo anti-PD-L1 blockade. Y10 mice chronically infected with Friend virus were injected i.p. every other day for 7 or 10 total injections with 250 μg functional grade 10F.9G2 (BioXCell). Control mice were concurrently given 250 μg rat IgG (BioXCell). Tissues were harvested the second day following the final injection.

RNAseq. RNA was isolated as described above for the in vitro suppression and RT-PCR assays. Total RNA was purified using phenol-chloroform extraction followed by RNeasy MinElute Cleanup Kit as per manufacturers instructions. cDNA libraries were prepared using the Ovation RNA-Seq system V2 by Nugen, Nextera DNA Library Preparation Kit for Illumina, and Nextera dual index (i7 and i5) adapter sequences. RNAseq was performed by the Stanford Functional Genomics Facility (Illumina NextSeq). Computing for this project was perfomed on the Stanford Sherlock cluster. Stanford Functional Genomics Facility extracted and generated FASTQ files for each sample, distinguished by the Nextera dual index adapters. Raw reads were trimmed for base call quality (phred>=21) and adapter sequences using Skewer. Processed reads were aligned to mm10 and read counts were generated using STAR 2.5.3a. The R package ‘DESeq2’ was used to normalize read counts, perform differential gene expression analysis, and generate the heat map. Transcripts were considered differentially expressed if they had a Benjamini-Hochberg adjusted p-value <0.1.

HCV cohort. PBMC, Plasma, and Serum were studied in fifteen HCV-infected patients. Ten patients underwent at least one previous treatment with interferon, the other five were treatment naïve. PBMC, Plasma, and Serum was collected in ten non-infected patients as a control. Patients provided written informed consent for research testing that complied with the ethical regulations under protocol 13859 by the Stanford University Institutional Review Board.

### HCV Patient Characteristics

| ID | Previous IFN | Genotype | Liver transplant waitlist | Sex |
|---|---|---|---|---|
| 2 | Yes | 1 | No | Male |
| 4 | Yes | 1 | No | Female |
| 7 | No | 2 | No | Female |
| 8 | Yes | 2 | No | Female |
| 9 | Yes | 1 | No | Male |
| 12 | No | 2 | No | Female |
| 13 | No | 1 | Yes | Female |
| 14 | Yes | 1 | No | Male |
| 20 | Yes | 1 | No | Male |
| 22 | Yes | 1 | No | Female |
| 27 | Yes | 1 | No | Male |
| 29 | Yes | 1 | Yes | Male |
| 30 | No | 4 | No | Female |
| 35 | No | 1 | No | Male |
| 38 | Yes | 1 | No | Male |

Phospho-CyTOF sample processing and staining. Cryopreserved PBMCs stored at −180° C. were thawed in warm RPMI medium supplemented with 10% FBS, benzonase, and a penicillin streptomycin mixture (complete RPMI). Cells were transferred into serum-free RPMI medium containing 2 mM EDTA and benzonase, incubated with cisplatin for one minute, and immediately quenched with four volumes of complete RPMI. Then, one million cells per sample were transferred into complete RPMI and rested for 30 minutes at 37° C. Following this rest period, cells were fixed in PBS with 2% paraformaldehyde (PFA) at room temperature for 10 minutes. Cells were then washed 2× with CyFACS buffer and barcoded using platinum- and palladium-labeled CD45 conjugates. Following barcoding, samples were combined for surface marker staining, performed at room temperature for 1 hour. Subsequently, cells were washed and permeabilized in MeOH at −80° C. overnight. The next day, cells were washed and incubated with the intracellular cytokine cocktail at room temperature for one hour. DNA stain was performed for 20 minutes with iridium (191/193) in PBS with 2% PFA at room temperature. Finally, cells were washed 2× with CyFACS buffer and then twice with MilliQ water before data acquisition on the CyTOF2 instrument. Data was de-barcoded and manually analyzed on Cytobank.

In vitro stimulation of human CD8 T cells. 96-well flat bottom tissue culture plates were coated with anti-human CD3 (1 ug per ml) and anti-human CD28 (3 ug per ml) in PBS for 2 hours. One million PBMCs were then plated in each well and stimulated (or left unstimulated in T cell media of RPMI containing supplementation with 50 units per ml IL-2 from Peprotech) for 5 days. Cells were then stained and analyzed by flow cytometry.

Linear regression modeling. For the estimation of regression coefficients, we iteratively conducted multiple linear regressions with the scalar dependent variables set as the median expression of each marker in the major PBMC cell subsets, and the explanatory variables set as age, sex, history of previous IFN treatment, history of cirrhosis, history of transplantation, sofosbuvir treatment regimen, HCV genotype, and HCV infection status. Regression coefficients with values different from zero at a false discovery rate (FDR) threshold of q<0.05 were considered significant.

1. A method of determining whether a cell or cell population is responsive to a therapeutic regimen to activate and expand exhausted CD8+ T cells, the method comprising:
assaying a cell sample from an individual to determine if functional CD8⁺, SIRPα+ functional T cells are present.

2. The method of claim 1, comprising contacting a population of T cells with an affinity agent for SIRPα, and detecting the presence of bound agent.

3. The method of claim 2, further comprising detecting the presence of PD-1 and/or CD8 on the T cells.

4. The method of claim 1 wherein the biological sample is one or more of a swab, skin sample, blood sample, a biopsy sample, a fine needle aspirate.

5. The method of claim 1, wherein the cell sample is obtained from an individual with cancer.

6. The method of claim 1, wherein the cell sample is obtained from an individual with a chronic infection.

7. The method of claim 1, wherein functional T cells positive for SIRPα and one or more inhibitory receptors selected from PD-1, CTLA-4, LAG-3, TIM-3.

8. The method of claim 1 wherein the functional CD8+ T cells are specific for a tumor antigen or a pathogen antigen.

9. The method of claim 1, wherein the patient is treated with a regimen to expand CD8⁺, SIRPα+ functional T cells.

10. The method of claim 9, wherein the regimen comprises blockade of inhibitory receptors.

11. The method of claim 10 wherein the patient is treated with a regimen comprising blockade of PD-1/PD-L1.

12. The method of claim 11 wherein the regimen comprises administering an effective dose of an antibody that blocks PD-1/PD-L1.

13. The method of claim 9, further comprising obtaining a patient sample following the regimen, to determine if there is an expansion of CD8⁺, SIRPα⁺ functional T cells.

14. A method of isolating functional PD-1⁺ CD8+ T cells, the method comprising isolating from a population of such T cells, cells that co-express SIRPα.

15. The method of claim 14, wherein the T cells thus isolated are analyzed for antigenic specificity to identify appropriate antigens for stimulation.

16. The method of claim 14, wherein the T cells thus isolated are stimulated and expanded in culture.

17. The method of claim 16, wherein the expanded T cell population is reintroduced into the individual for therapeutic purposes.

18. The method of claim 15, wherein the cognate antigen is provided in combination with a regimen in order to stimulate the CD8⁺, SIRPα⁺ functional T cells.
